(19) United States
(12) Patent Application Publication
(10) Pub. No.: US 2026/0338649 A1
(43) Pub. Date: Oct. 29, 2026

(54) BUOYANCY ACTIVATED CELL SORTING AND SCALABLE PROTEIN PURIFICATION WITH ENGINEERED GAS VESICLES

(71) Applicants: William Marsh Rice University, Houston, TX (US); Board of Regents, The University of Texas System, Austin, TX (US)

(72) Inventors: Jiaozhi LU, Houston, TX (US); Eugene CHUNG, Houston, TX (US); Luke HAINES, Houston, TX (US); Zongru LI, Houston, TX (US); Chia-Yu HO, Houston, TX (US); Cassian YEE, Houston, TX (US); Shailbala SINGH, Houston, TX (US); Xueqian WANG, Houston, TX (US)

(73) Assignees: William Marsh Rice University, Houston, TX (US); Board of Regents, The University of Texas System, Austin, TX (US)

(21) Appl. No.: 19/122,227

(22) PCT Filed: Oct. 20, 2023
(86) PCT No.: PCT/US2023/077430

§ 371 Date: Apr. 17, 2025

Related U.S. Application Data
(60) Provisional application No. 63/417,928, filed on Oct. 20, 2022.

(51) Int. Cl. G01N 1/34

(52) U.S. Cl. CPC G01N 1/34

(57) ABSTRACT

In one aspect, the present disclosure describes methods which may be used to purify target proteins from sample mixtures. The methods disclosed herein may also be used to purify or sort cells from sample mixtures.

### PRIORITY CLAIM

This application claims benefit of priority to U.S. Provisional Application Ser. No. 63/417,928, filed Oct. 20, 2022, the entire contents of which are hereby incorporated by reference.

### SEQUENCE LISTING INCORPORATION

This application contains a Sequence Listing XML, which has been submitted electronically and is hereby incorporated by reference in its entirety. Said XML Sequence Listing, created on Oct. 20, 2023, is named RICEP0114WO.xml and is 6,269 bytes in size.

### BACKGROUND

### 1. Field

This disclosure relates to the fields of biology, biochemistry, molecular biology, biomanufacturing, biotechnology, and medicine. In particular, new methods related to purifying or isolating enzymes, proteins, and cells are disclosed.

### 2. Related Art

Cell-based therapies, such as autologous transplants and engineered T-cell therapies like CAR-T treatments, is a rapidly expanding class of therapeutics (Bashor et al., 2022). Essential to the success of these therapies is the ability to sort and modify specific cell populations consistently and efficiently (Lipsitz el al., 2016). As such, effective and economical cell sorting techniques are critical. Magnetic-activated cell sorting (MACS) is a widely used technique that utilizes magnetic microbeads modified with antibodies to target cellular antigens (Miltenyi el al., 1990; Wang et al., 2022; Wang et al., 2023; Frenea-Robin & Marchalot, 2022). In the presence of a magnetic field, cells bound to these microbeads are isolated. Despite its advantages, including high cell recovery and purity, concerns exist regarding the cost of the magnetic bead reagents and potential downstream cellular effects from the magnetic labeling (Frenea-Robin & Marchalot, 2022).

Fluorescence-activated cell sorting (FACS) is another method that sorts cells tagged with fluorescent markers based on their fluorescence intensity and light-scattering properties via a flow cytometry system (Hu et al., 2016). However, FACS sometimes faces challenges in throughput due to droplet generation rates and scalability (Hu et al., 2016). Buoyancy-activated cell sorting (BACS) employs synthetic microbubble-based reagents, showcasing the potential of buoyancy-based processes for cell sorting (Liou et al., 2015). A common challenge across these techniques is the significant cost associated with the production of recombinant proteins, especially antibodies used for surface-antigen specific sorting (Weil et al., 2017). The purification and modification expenses of recombinant protein reagents play a critical role in elevating overall manufacturing costs (Palani et al., 2023).

Protein purification is a fundamental step in many industrial sectors, such as enzyme catalysis for commodity chemicals (Roe, 2001; Scopes, 1993; Deutscher, 1990), biopharmaceuticals (Rathore et al., 2018; Jozala et al., 2016; Dos Santos et al., 2017; Hanke & Ottens, 2014), agricultural and food industry (Liu et al., 2020; Peydayesh & Mezzenga, 2021), and bioremediation (Viswanath et al., 2014; Cheung & Gu, 2007; Chakroun et al., 2010), with a combined global market of over billion dollars. Current in-lab and in-industry methods include a variety of techniques such as chromatography, electrophoresis, precipitation, and solubilization (Roe, 2001; Deutscher, 1990; Janson, 2012; Linhult et al., 2005; Flickinger, 2013; Harrison, 1993; Franzreb et al., 2007). Chromatography-based methods are the most used techniques; however, these methods can be time-consuming, require specialized equipment, and often result in low yields (Ghosh, 2022; Gomari et al., 2020). Meanwhile, methods that rely on phase separation, such as precipitation (Labrou, 2014; Burgess, 2009) and oil droplets (Leptihn et al., 2013; Yang & Sagis, 2021), are relatively simple and can be used for large-scale and consistent purification. However, they often lead to protein loss and low purity. Thus, there is a need to develop new techniques that can combine the scalability of phase separation methods and the selectivity of chromatography.

The current gold standard in laboratory scale peptide purification is affinity chromatography, such as using Ni-NTA column to purify a poly-histidine tagged protein of interest (POI). This technology usually handles samples which have a volume of less than 100 milliliter. At the industrial scale, which may require purification of samples which have a volume of several tens of liters, chromatography becomes cost-prohibitive. For all of these reasons, methods are required which enable large-scale purification of protein targets.

Ex vivo cell manufacturing is a critical step and represents a large market in cell-based therapy. Existing cell manufacturing workflows include multiple positive-cell selection steps that pose labor-intensive bottlenecks. Present manufacturing methods employ magnetic activated cell sorting (MACS), which involves attaching antibody-tagged target cells to magnetic beads and retaining them in a magnetic column. Because of the magnetic column and the required setup, these methods can really only allow for separations in the x-y geometry. Therefore, methods that allow for bulk cell sorting, such as by expanding the x-y geometry of the cell sorting vessel, would represent an improvement to the state of the art. Therefore, there remains a need for cell sorting methods which are more amenable to industrial scale process and production. This invention was funded in part by the Robert A. Welch Foundation under Welch Grant No. C-2069-20210327.

### SUMMARY

As provided herein, the present disclosure relates to methods of purifying proteins using genetically engineered gas vesicles or isolating cells using formulated nano- and micro-structures containing gas vesicles. In some embodiments, the gas vesicles disclosed herein display specific markers, peptides, or proteins which enable selective binding to target cells or proteins. In some embodiments, the gas vesicles comprise proteins which are pre-translationally modified, which provides the advantage of reducing processing steps or simplifying isolation of target cells or proteins.

The use of labeled GVs as disclosed herein provides a method to capture proteins, for example for density-based bioprocesses or functional modifications for targeted applications, with improved control or improved predictability. In some embodiments, the present disclosure provides methods for isolating or purifying target proteins that are improved relative to methods known in the art, such as nickel affinity chromatography. In some embodiments, the present disclosure provides methods for isolating and purifying target proteins that have improved yield compared to methods known in the art, such as nickel affinity chromatography. In some embodiments, the present disclosure provides methods for isolating and purifying target proteins that have improved purity compared to methods known in the art, such as nickel affinity chromatography. In some embodiments, the present disclosure provides methods that facilitate scalable isolation or purification of target proteins or cells for industrial applications. In some embodiments, the methods disclosed herein involve flotation of gas vesicle compositions for extended periods of time under normal gravity or over long distances, thereby mitigating the need for specialized equipment that may be required in similar methods known in the art.

The present invention utilizes the buoyancy of the gas vesicles used in the methods disclosed herein to adjust the overall density of the target protein or target cell once it is bound to the gas vesicle, thereby facilitating improved or simplified separation by centrifugation or other density-based isolation or purification methods. The gas vesicles of the present disclosure, which comprise a protein shell, a hydrophobic interior, and a hydrophilic exterior, have the advantageous combination of high physical stability and ability to repel water (Pfeifer, 2012; Walsby, 1994).

In some embodiments, the present methods may be used for cell sorting, such as T-cell sorting. In some embodiments, the present methods comprise using density-based fractionation to sort target cells or proteins. The present methods may involve a variety of gas vesicle labeling schemes to bind target protein or target cells. In some embodiments, the present methods use gas vesicles comprising SpyTag with the SpyTag/SpyCatcher protein ligation systems. In other embodiments, streptavidin-GVs bind to biotinylated targets, such as biotinylated antibodies. The present methods may involve modular functionalization of gas vesicles with SpyCatcher fusion proteins. In some embodiments, the present methods facilitate T-cell separation. In some embodiments, the presently disclosed methods provide a more streamlined and cost-effective approach to cell sorting over methods known in the art.

Details on these aspects and more are provided below and in the sections that follow.

In some embodiments, the present disclosure provides methods of purifying a target protein from a sample comprising:
(A) obtaining a collection of labeled gas vesicle composition, wherein gas vesicles comprising one or more proteins on the surface of the gas vesicles and the one or more proteins have been modified with one or more affinity tag to form a labeled gas vesicle composition; and
(B) contacting the collection of labeled gas vesicle composition with a sample containing the target protein for a first time period; wherein the first time period is sufficient for the labeled gas vesicle composition and the target protein to bind to form a captured target protein-gas vesicle composition; and
(C) allowing the components of the sample containing the captured target protein-gas vesicle composition to equilibrate for a second time period; and
(D) isolating the captured target protein-gas vesicle composition from the sample mixture.

In some embodiments, the captured target protein-gas vesicle composition forms a floating layer in the sample mixture. In some embodiments, the floating layer forms during the first or second time period.

In some embodiments, the methods comprise a target protein on the surface of a cell type in the sample mixture. In some embodiments, the cell is a lymphatic cell, such as a T cell. In some embodiments, the cell is derived from a patient.

In some embodiments, the target protein is present on a single cell type in the sample mixture. In other embodiments, the target protein is present on two or more cell types in the sample mixture.

In some embodiments, the present disclosure provides for methods wherein the collection of labeled gas vesicle compositions comprises two distinct sets of labeled gas vesicle compositions; wherein
(i) the first set of labeled gas vesicle compositions comprise a first affinity tag;
(ii) the second set of labeled gas vesicles compositions comprise a second affinity tag; and

wherein the first affinity tag and the second affinity tag are complementary.

In some embodiments, the labeled gas vesicle compositions further comprise a cell-specific marker. In further embodiments, the cell-specific marker is attached to an affinity tag identical to the first affinity tag of the first set of labeled gas vesicle compositions.

In some embodiments, contacting the labeled gas vesicle compositions with the sample mixture occurs in two steps, wherein
(iii) the first step comprises contacting the first set of labeled gas vesicle compositions with the sample mixture; and
(iv) the second step comprises contacting the second set of labeled gas vesicle compositions with the sample mixture.

In some embodiments, the second set of labeled gas vesicle compositions is present in excess relative to the first set of labeled gas vesicle compositions. In some embodiments, at least one of the affinity tags of the present methods is a peptide or protein. In some embodiments, at least one of the affinity tags is a peptide. In some embodiments, at least one of the affinity tags is a protein. In further embodiments, at least one of the affinity tags is an antibody, such as an antibody against the target protein. In still further embodiments, the target protein is expressed on the surface of a cell. In some embodiments, the antibody is an anti-CD3 antibody. In other embodiments, the antibody is an anti-CD28 antibody. In some embodiments, at least one of the affinity tags is streptavidin. In some of the embodiments, at least one of the affinity tags is biotin. In some of the embodiments, at least one of the affinity tags is SnoopTag. In some embodiments, at least one of the affinity tags is SnoopCatcher. In some embodiments, at least one of the affinity tags is SpyTag.

In some embodiments of the present methods, the labeled gas vesicle composition and the target protein bind to form a covalent bond. In other embodiments, the labeled gas vesicle composition and the target protein bind to form a non-covalent bond.

In some embodiments of the methods disclosed herein, the first time period is from about 15 minutes to about 5 hours, such as about 1 hour. In some embodiments of the present methods, the second time period is from about 15 minutes to about 5 hours, such as about 1 hour or about 2 hours. In some embodiments, the second time period is about 5 hours.

In some embodiments, the method does not comprise centrifugation. In other embodiments, the method comprises centrifugation. According to some embodiments of the present methods, isolating the captured target protein-gas vesicle composition further comprises centrifugation. In further embodiments, the centrifugation is traditional centrifugation. In other embodiments, the centrifugation is density-gradient centrifugation. In some embodiments, the centrifugation is at 400×g. In some embodiments of the present disclosure, isolating the captured target protein-gas vesicle composition further comprises elutriation.

In some embodiments, the present methods further comprise separating the target protein from the captured target protein-gas vesicle composition following isolation from the sample mixture. In further embodiments, the separation occurs via cleavage by a protease, such as TEV protease. In still further embodiments, the separation occurs via intein self-splicing. In yet further embodiments, the intein is AI-CM mini intein. In some embodiments, the intein self-splices upon pH change. In further embodiments, the intein self-splices upon changing the pH from about pH 4 to about pH 7. In other embodiments, the intein self-splices upon temperature change.

In some embodiments, the present methods further comprise detecting the presence of the target protein. In further embodiments, the detection is a detection of the presence of a cell.

In some embodiments, the sample containing the target protein has a volume of about 10 L or greater. In further embodiments, the sample containing the target protein has a volume of about 100 L or greater. In still further embodiments, the sample containing the target protein has a volume of about 1000 L or greater. In yet further embodiments, the sample containing the target protein has a volume of about 10,000 L.

In one embodiment, methods of purifying proteins using affinity tag on buoyant nanostructures are disclosed for scalable production of biomolecules and enzymes, such as those used for commodity, biofuel or material manufacturing. In another embodiment, methods to formulate buoyant nano- and micro-structures with antibodies suitable for surface binding or being endocytosed by cells are disclosed for pharmaceutical applications such as those for in vitro cell therapy manufacturing.

It is contemplated that any method or composition described herein can be implemented with respect to any other method or composition described herein. For example, a compound synthesized by one method may be used in the preparation of a final compound according to a different method.

The use of the word “a” or “an” when used in conjunction with the term “comprising” in the claims and/or the specification may mean “one,” but it is also consistent with the meaning of “one or more,” “at least one,” and “one or more than one.” The word “about” means plus or minus 5% of the stated number.

Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 shows the genetic engineering of affinity tags onto the surface of gas vesicles.

FIG. 2 shows the modified density of cells expressing single and dual target proteins of interest (POI) based on degree of gas vesicle attachment for buoyancy-assisted cell sorting.

FIG. 3 illustrates the capture and purification of protein of interest (POI) based on affinity tag interaction and the buoyancy-based isolation of composition.

FIGS. 4A-4H show GV modification strategies and GV characterization. (FIG. 4A) Wild type (WT) GV structure displaying the annealing pattern of GvpC. (FIG. 4B) NHS-Streptavidin chemical modification of GVs. (FIG. 4C, FIG. 4D) GvpC replacement on WT GVs with modified and recombinantly expressed GvpC fused to SpyTag. (FIG. 4E) Covalent modification of GvpC-SpyTag with mWasabi-SpyCatcher fusion protein for surface decoration of GVs. (FIG. 4F) Covalent modification of GvpC-SpyTag with scFv-SpyCatcher fusion protein. (FIG. 4G) SDS-Page observation of ligation between SpyTag and SpyCatcher for GV samples replaced with a range GvpC-SpyTag replacement (BP-Buoyany Purification, mWasa-mWasabi, SpyC-SpyCatcher, SpyT-SpyTag). (FIG. 4H) Relative fluorescent intensity of GVs modified with decreasing amounts of GvpC-SpyTag after ligation with mWasabi-SpyCatcher, normalized to sample OD.

FIGS. 5A-5D provide evidence of the modified density of RAW 264.7 macrophages after incubation with GVs. (FIG. 5A) Photograph of cell samples when incubated with PBS (left) or GV at an OD₅₀₀=5 (right). (FIG. 5B) Percentage of cells recovered in the top fraction across a density series when treated with PBS (grey bar) or GV (blue bar). (FIG. 5C) Photograph of column devices used for density-media fractionation of top and bottom cells. Yellow arrow indicates a cell layer captured above the density media. Size of bottom fraction cell pellets are visualized (bottom) (FIG. 5D) Photographs of the GV treated cell samples and their separation between the bottom and top fractions.

FIGS. 6A-6G show that anti-CD3 scFv modified SpyTag GVs enrich CD4+ and CD8+ cells in the buoyant fraction. (FIG. 6A) SpyTag GVs are ligated with anti-CD3 scFv-SpyCatcher fusion protein. (FIG. 6B) Schematic of cell density reduction corresponding to level of GV binding, governed by CD3 surface antigen recognition. (FIG. 6C) Density column insert system used for isolation buoyant (up fraction) and pelleting (down fraction) cells after centrifugation. (FIG. 6D) Photograph of distinct layers observed in the density column after processing. (FIG. 6E) Percentage of T-Cells in unprocessed peripheral blood monocular cells (PBMCs), or in the up fraction after incubation with either PBS or CD3-GVs, separated with a 1.06 g/mL density media. (FIG. 6F) FSC/SSC profile show significant increases in SSC of GV-treated up fraction, particularly in the FSC cluster comprising lymphocytes. (FIG. 6G) Distribution of CD4+ or CD8+ T-Cells in the up and down fractions of PBMCs treated with or without GVs show enrichment of T-Cells.

FIGS. 7A-7B show the reference anti-CD3 scFv sequence validation with cell binding studies (FIG. 7A) Flow cytometry data comparing the staining distribution of anti-CD3 scFv-mWasabi fusion (top) compared to a commercially available, fluorophore modified anti-CD3 antibody (OKT3 clone). Number inlay reports % of cells staining above negative staining controls. (FIG. 7B) Concentration titration of anti-CD3 svFv-mWasabi for staining cells.

FIGS. 8A-8C illustrate cell density modification across a PBS—Ficoll-Paque density ladder. (FIG. 8A) Percent depletion of cells in the bottom pelleting cell fraction when treated with and without GVs. (FIG. 8B) CD14+ monocyte burden in up and down fractions for cell samples separated using 1.04 and 1.06 g/mL density media. Number inlay indicated percentage of CD14+ cells in the fraction. (FIG. 8C) CD14+ monocyte burden in CD3-GV treated cell fractions.

FIGS. 9A-9B show validation of Streptavidin GV modification. (FIG. 9A) SDS-Page of unmodified GVs (Ana GV), GVs after Lightning-Link chemical modification with Streptavidin, and after two rounds of buoyancy purification (BP) indicate ligation of Streptavidin to GVs. (FIG. 9B) TEM imaging of the clustered and unclustered states of biotin modified GVs and streptavidin-modified GVs.

FIGS. 10A-10E provide evidence that streptavidin-GVs can be used according to the present methods with off-the-shelf biotinylated antibodies. (FIG. 10A) Schematic of biotinylated antibody and streptavidin-modified GVs as a 2-part cell labeling kit. (FIG. 10B) Labeling scheme of cells with a primary antibody and secondary GV binding. (FIG. 10C) FSC/SSC analysis of untreated GVs (w/o GV) or with a biotinylated anti-CD3 commercial antibody followed by streptavidin-GVs. Inlay shows the CD14+ monocyte burden with the events with high GV binding. (FIG. 10D) CD14+ monocyte burden in up and down fractions for PBMCs separated using 1.04 and 1.06 g/mL density media after treatment with PBS. (FIG. 10E) CD14+ monocyte burden for PBMCs separated after treatment with antibody+GV.

FIGS. 11A-11C illustrate the Ni-NTA purification of POI from E. coli expression. (FIG. 11A) Purification of GvpC-SpyTag-His. (FIG. 11B) Purification of mWasabi-SpyCatcher-His. (FIG. 11C) Purification of anti-CD3 scFv-SpyCatcher-His. WC:Whole Cell Lysate, S:Supernatant, FT: Flow-Through, E:Elution, W:Wash

FIG. 12 provides the results of an assay to determine GvpC solubility. GvpC is depleted from GVs after buoyancy purification in the presence of 0-6M urea.

FIG. 13A-13K provide evidence via affinity tags insertion screening that SnoopTag and Strep-tag II can be introduced to the C-terminus of shell protein GvpB without disrupting GV assembly. FIG. 13A: The architecture of the pNL29 operon. Shell protein genes were labeled deep blue. FIG. 13B: Representative TEM images of pNL29 GVs after unclustering by 6M urea treatment. Scale bar=500 nm. c, Cryo-EM structure of the GvpB monomers, and the left-handed helical GV shell structure formed from assembled monomers. Schematic shows a helical GV shell lattice with closely-packed GvpB monomers, featuring 1.2 nm lateral and 4.9 nm helical spacing¹. FIG. 13D, FIG. 13E: Schematic of the genetic addition of linkers and tags to GvpB's C-terminus. Upon expression and assembly, GVs are expected to have a surface with affinity tags. FIG. 13F: A table of the major shell protein with affinity tags and linkers appended on C-terminus constructs, characterizations of their expression and assembly in E. coli. For GV expression and assembly, single plus signs (+) represent that a barely sufficient amount of intact GVs could be observed by naked eyes after cell lysis, and triple plus signs (+++) indicate the number of GVs was enough for both TEM and DLS characterizations, and minus signs (−) represent GVs were not detected. FIG. 13G, FIG. 13H: Representative TEM images of pNL29 GVs with shell protein GvpB genetically modified with SnoopTag (GvpB::SnoopTag) and StrepTag II (GvpB::StrepTag II). Scale bar=500 nm. FIG. 13I, FIG. 13J: Representative DLS measurement of the hydrodynamic diameter and mean hydrodynamic diameter (n=4 biological replicates) of GV variants. WT: wildtype unmodified pNL29 GVs; SnoopTag: pNL29 GVs with GvpB genetically modified with SnoopTag (GvpB::SnoopTag); StrepTag II: pNL29 GVs with GvpB genetically modified with Strep-tag II (GvpB::Strep-tag II). Error bars represent mean±standard deviation (STDEV). **p<0.01; ***p value<0.001. FIG. 13K: Schematic of applying genetically tagged GVs on target recombinant protein purification.

FIGS. 14A-14F show that 6M urea treatment is not able to uncluster tagged GVs. FIG. 14A, FIG. 14B: Representative TEM images show wildtype pNL29 GVs remaining clustered (FIG. 14A) post-purification from E. coli and transitioning to an unclustered state (FIG. 14B) after treatment with 6M urea. Scale bar=500 nm. FIGS. 14C-14F Representative DLS measurement of the hydrodynamic diameter and mean hydrodynamic diameter (n=4 biological replicates for WT and Strep-tag II GVs, and n=5 biological replicates for SnoopTag GVs) of GV variants before and after 6M urea treatment. Error bars represent mean±standard deviation (STDEV).

FIGS. 15A-15E present interaction studies of tagged GVs with their affinity tag binding partners, which indicate that surface-expressed tags on GVs remain functional. FIG. 15A: Schematic of the assay using fluorescence-labeled binding partners to confirm the functionality of affinity tags on the surface of GVs. 1: Tagged GVs were loaded into a tube with samples of fluorescence-labeled binding partners of affinity tags. 2: The samples were placed on a rotating mixer for 1-hour incubation at room temperature to allow for interactions between the tags on the GVs and the binding partners. Immediately following incubation, the fluorescence intensity of the mixed sample was measured. 3: The samples were then centrifuged at 400×g for 10 minutes. 4: Post-centrifugation, GVs with bound fluorescence-labeled binding partners buoyantly floated to the liquid surface. A syringe was used to remove the liquid and any unbound binding partners located beneath the GVs. 5: The samples were resuspended in 1×PBS, and the fluorescence intensity of the resuspended sample was measured immediately. 6: This process (steps 3-5) was repeated at least three times, or until the fluorescence intensity of the resuspended samples showed no significant change. FIGS. 15B-15E:The percentage of retained fluorescence intensity on the GVs (n=3 biological replicates), relative to the initial fluorescence intensity measured right after sample incubation, along with fluorescence images of the samples after the final round of washing and centrifugation. PBS: only 1×PBS presented in the tube without any samples. Error bars represent mean±standard deviation (STDEV).

FIGS. 16A-16B show the corrections between protein of interest concentrations and corresponding fluorescence values. The y-axis depicts the fluorescence readouts corrected for blanks, while the x-axis shows protein concentrations. Displayed measurements include 150 μL of purified His₆-tag::SnoopCatcher::sfGFP (FIG. 15A) and His₆-tag::SnoopCatcher::miniSOG (FIG. 15B).

FIGS. 17A-17B Fluorescence values of the underlying liquid removed from each round of buoyancy isolation. The y-axis represents the fluorescence values adjusted for blanks, and the x-axis denotes each buoyancy isolation round. The presented measurements come from the underlying liquid syringed out during the functionality test of the affinity tags assay for purified SnoopTag GVs (FIG. 17A) and Strep-tag II GVs (FIG. 17B) (n=3 biological replicates). Error bars represent mean±standard deviation (STDEV).

FIGS. 18A-18H provide a side-by-side comparison of recombinant protein purification using HisTag/Ni-NTA immobilized metal affinity chromatography (IMAC) versus tagged GV-associated buoyancy-assisted affinity chromatography (BAAC). FIG. 18A: The architecture of the His₆-tag::SnoopCatcher::miniSOG operon, TEV cleavage site (ENLYFQ|S (SEQ ID NO:5)) was inserted between miniSOG and SnoopCatcher. FIG. 18B: Schematic of how the target protein, miniSOG, was ‘eluted’ from the surface of tagged GVs through the action of TEV protease cleaving at the recognition site. FIG. 18C: Schematic of His-tag/Ni-NTA immobilized metal affinity chromatography (IMAC) and tagged GVs buoyancy-assisted affinity chromatography (BAAC). FIG. 18D, FIG. 18E: The percentage of retained fluorescence intensity on the GVs, relative to the initial fluorescence intensity measured right after sample incubation, along with fluorescence images of the samples after the incubation with TEV protease followed by centrifugation and buoyancy purification to syringe out the sub-natant (fluorescence content boxed). W1 to W4: Fluorescence intensity remained on GVs percentage after 1st to 4th 1×PBS resuspension. PE: Fluorescence intensity remained on GVs percentage after incubation with TEV protease, followed by buoyancy purification and 1×PBS resuspension (n=6 biological replicates). FIG. 18F: Percentage of miniSOG purified from tagged GVs BAAC or His-tag/Ni-NTA IMAC relative to cell lysate. For the tagged GVs method, % of POI purified=100×(fluorescence intensity of miniSOG after removing the TEV protease)/(fluorescence intensity of miniSOG in the supernatant of cell lysis). For the Ni-NTA method, % of POI purified=100×(fluorescence intensity of miniSOG of elution from Ni-NTA)/(fluorescence intensity of miniSOG in the supernatant of cell lysis) (n=6 biological replicates for tagged GVs method and n=3 biological replicates for Ni-NTA method). FIG. 18G: SDS-PAGE gel for miniSOG purified from E. coli clarified lysate by tagged GVs BAAC. Pre: pre-induction; Post: post-induction; Pellet and Sup.: pellet and supernatant after cell lysis and centrifugation; W1, W2, W3, and W4: the washing fractions removed by syringe; P_{TEV}: sub-natant of the sample after TEV protease incubation and centrifugation; E: P_{TEV} after removing the TEV protease; Purity of E was determined by densitometry (right), gray area under the red line represents background intensity. FIG. 18H: SDS-PAGE gel for miniSOG purified from E. coli clarified lysate by Ni-NTA/His-tag IMAC. Pre: pre-induction; Post: post-induction; Pellet and Sup.: pellet and supernatant after cell lysis and centrifugation; Flow-Thru: flow-through of Ni-NTA chromatography; W1, W2, and W3: the washing fractions of Ni-NTA chromatography; Elution 1-3: elution fractions of Ni-NTA-chromatography; P_{TEV}: after TEV protease incubation; E: P_{TEV} after removing the TEV protease; Purity of E was determined by densitometry (right), gray area under the red line represents background intensity. Error bars represent mean±standard deviation (STDEV).

FIGS. 19A-19G illustrate “One-step” and “equipment-free” buoyancy-assisted recombinant protein purification. FIG. 19A: Schematic of “One-Step” buoyancy-assisted recombinant protein purification procedures. E. coli cultures containing tagged GVs and the target protein were lysed together. This was followed by centrifugation to separate the target protein bound to tagged GVs (1), the soluble unbound target protein (2), and inclusion bodies (3). The supernatant, containing soluble unbound target protein and inclusion bodies, was removed via syringe. The tagged GVs bound with target protein were then resuspended in 1×PBS. This buoyancy purification process is repeated three times, or until the supernatant is clear and contains only the target protein bound to tagged GVs (4). The sample is then resuspended and incubated with TEV protease at 30 °C for 12 hours. After a final centrifugation, some target proteins remain on the surface of tagged GVs (5), while others are released and become soluble in the supernatant (6). FIG. 19B, FIG. 19C: SDS-PAGE and Western blot results indicates the final elution step in the “One-Step” buoyancy-assisted recombinant protein purification process. Lane 1 contains the supernatant from samples post-TEV protease incubation; Lane 2 shows the flow-through (Elution 1) following TEV protease with His₆-tag removal via Ni-NTA magnetic beads; Lane 3 displays the wash (Elution 2) after rinsing the Ni-NTA magnetic beads; Lane 4 is the final elution from the Ni-NTA magnetic beads. Grey arrows point to either the TEV protease with His₆-tag (in both SDS-PAGE and Western blot) or GvpB with His₆-tag::SnoopCatcher (evident as smearing in the Western blot). Green arrows highlight sfGFP present in Lanes 2 and 3. d, The amount of target protein, sf GPF, yielded from two rounds of ‘elution’ procedures. sfGFP amount was calculated from fluorescence intensity. FIG. 19E, FIG. 19F: Schematic of the time-lapse photography setup for analyzing the buoyant flotation speed of GVs: The setup captured initial and final states of well-suspended, tagged GVs or GVs bound to fluorescent proteins in a 10 cm long Corning tube. The first photo (left in FIG. 19F) shows the GVs at the beginning of the time-lapse sequence, and the final photo (right in FIG. 19F) depicts them after they have floated to the surface of the liquid, a process that takes approximately 5.3 hours. FIG. 19G: Grey intensity measurements were taken from selected regions of interest on tubes containing GVs over a 5-hour period. The resulting data was fit to a one-phase decay nonlinear model, as represented by the equation and R² displayed in the figure (n=3, biological replicates). Error bars represent mean±standard deviation (STDEV).

FIGS. 20A-20G show a side-by-side comparison of sfgGFP purification using HisTag/Ni-NTA immobilized metal affinity chromatography (IMAC) versus tagged GV-associated buoyancy-assisted affinity chromatography (BAAC). FIG. 20A, FIG. 20B: The percentage of retained fluorescence from sfGFP intensity on the GVs, relative to the initial fluorescence intensity measured right after sample incubation (FIG. 20A), along with fluorescence images of the samples (FIG. 20B) after the incubation with TEV protease followed by centrifugation and buoyancy purification to syringe out the sub-natant (fluorescence content boxed). W1 to W4: Fluorescence intensity remained on GVs percentage after 1st to 4th 1×PBS resuspension. PE: Fluorescence intensity remained on GVs percentage after incubation with TEV protease, followed by buoyancy purification and 1×PBS resuspension (n=5 biological replicates). Error bars represent mean±standard deviation (STDEV). FIG. 20D, FIG. 20E: Percentage (FIG. 20D) and amount (FIG. 20E) of sfGFP purified from tagged GVs BAAC or His-tag/Ni-NTA IMAC relative to cell lysate. For the tagged GVs method, % of POI purified=100×(fluorescence intensity of sfGFP after removing the TEV protease)/(fluorescence intensity of sfGFP in the supernatant of cell lysis). For the Ni-NTA method, % of POI purified=100×(fluorescence intensity of sfGFP of elution from Ni-NTA)/(fluorescence intensity of sfGFP in the supernatant of cell lysis) (n=5 biological replicates). sfGFP amount was calculated from fluorescence intensity. FIG. 20F: SDS-PAGE gel for sfGFP purified from E. coli clarified lysate by tagged GVs BAAC. Pre: pre-induction; Post: post-induction; Pellet and Sup.: pellet and supernatant after cell lysis and centrifugation; W1, W2, W3, and W4: the washing fractions removed by syringe; P_{TEV}: sub-natant of the sample after TEV protease incubation and centrifugation; E: P_{TEV} after removing the TEV protease; Purity of Elution was determined by densitometry (right), gray area under the red line represents background intensity. FIG. 20G: SDS-PAGE gel for concentrated sfGFP after removing the TEV protease by Ni-NTA/His-tag IMAC or SnoopTag GVs BAAC. Each purity was determined by densitometry (right), gray area under the red line represents background intensity.

FIG. 21 illustrates the POI lost in buffer exchange procedure for His-tag/Ni-NTA purification. The percentage of miniSOG and sfGFP lost after the buffer exchange procedure was conducted to remove high concentrations of imidazole from the elution steps in His-tag/Ni-NTA purification is calculated as: % of POI lost=100×(fluorescence intensity of POI before buffer exchange−fluorescence intensity of POI after buffer exchange)/(fluorescence intensity of POI before buffer exchange) (n=3 biological replicates for miniSOG and n=5 biological replicates for sfGFP). The total volume of POI remained consistent throughout. Error bars represent mean±standard deviation (STDEV).

### DESCRIPTION OF ILLUSTRATIVE EMBODIMENTS

The present disclosure relates to the development of methods for isolation of cells utilizing buoyant microbubbles. The microbubbles dissipate in minutes in solution, require gentle work-up protocols, and are limited with regard to the multiplex capability of the technology.

Provided herein are methods that have been demonstrated to purify target cells from mixed samples. In some embodiments, the target proteins are found on the surfaces of cells. Therefore, in some embodiments the methods of the present disclosure have been demonstrated to purify or sort cells from mixed samples via buoyancy assisted cell sorting (BACS). The methods described herein require low-speed centrifugation, elutriation, or gravity flotation, in contrast to harsher and lengthier conditions required by similar technology known in the art. Furthermore, the compositions described in the presently disclosed methods exhibit improved stability over those of similar technologies.

In some embodiments, the present methods provide for a greater degree of control in target cell or target protein capture in comparison to known methods. For example, traditional magnetic microbead-based cell sorting techniques are a binary yes or no in how cells are captured. A single antibody-modified bead needs to be bound to a target cell for it to be retained. On the other hand, a plurality of GVs, such as thousands, are required to modify the density of a single cell, and this can be influenced by label density on the surface of the GV, the use of multiple types of labels, changing the GV structure/volume, or employing formulated versions that are clustered.

Described herein are methods that may be used to sort a mixed population of mammalian cells during cell manufacturing. In an improvement over known methods of cell-sorting, the methods of the present disclosure do not require an external selection force, such as a magnetic column.

According to some embodiments of the present disclosure, GVs are purified and are labeled to induce protein-protein linkages with either antigen-specific tags or other GVs by way of biotin-streptavidin interactions. In some embodiments, labeled GVs may be incubated with a heterogenous cell population and bind to sub-populations of interest that are tagged with antibodies or other antigen-specific biomolecules, such as nanobodies or aptamers. In some embodiments, the buoyant force acting on the GVs provides upward lift to float the GV-cell complexes to the surface while untargeted cells sediment. These and more details will be discussed in more detail below.

The present disclosure also relates to the development of methods for isolation of proteins and enzymes in the industry for manufacturing commodities. The current methods such as large-size chromatography, protein secretion, non-specific binding of cells to bubbles are either cost-inhibitive or have limitations.

Described herein are methods that have been demonstrated to purify target proteins from mixed samples of cell lysates. These and more details will be discussed in more detail below.

### I. GAS VESICLES AND BUOYANCY ASSISTED PURIFICATION

Gas vesicles (GVs) are protein shell nanobubbles that are natively produced in Anabaena flos-aquae or Haloarchae salinarum, or heterologously in Escherichia coli. The gene cluster of GVs for E. coli expression includes one major shell protein, gvpB, the basic construct of GVs, and the other ten accessory proteins, gvpRNFGLSKJTU (SEQ ID NO:6). The methods of the present disclosure provide for at least one modification of at least one shell protein of a GV. In some embodiments, gvpB is modified. In some embodiments, gvpA is modified. In some embodiments, gvpC is modified. Modification of any of the ten accessory proteins is also contemplated. The present methods may also involve recombinant version of the shell proteins. For example, the GV shell protein sequences can be rationally modified to introduce additional functionalities or alter GV geometries, rigidity, and clustering state. Recombinant shell proteins can include genetically fused elements (e.g. tags) that can be separately expressed, purified, and swapped interchangeably onto wildtype GVs. GVs can also be assembled using recombinant expression by transforming engineered variants of the gvp gene cluster that includes the structural shell proteins (GvpA, GvpC), and the required accessory proteins for GV formation in a non-native host.

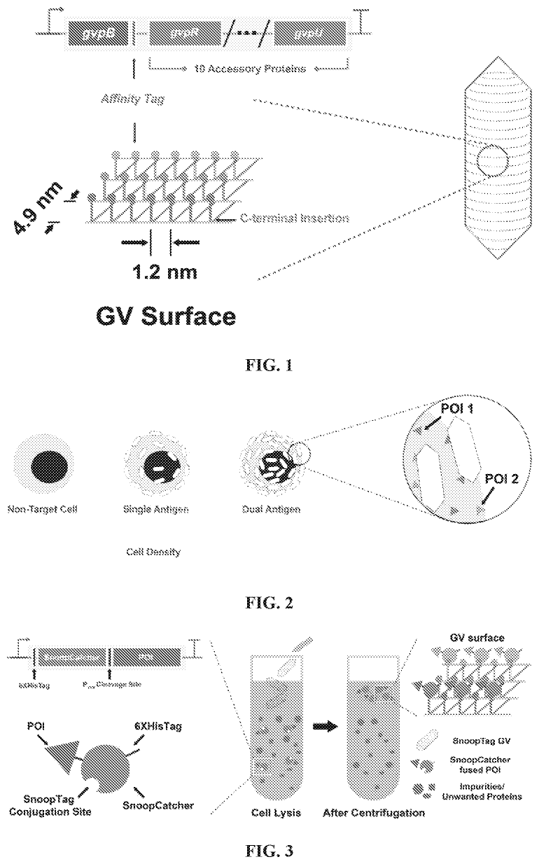

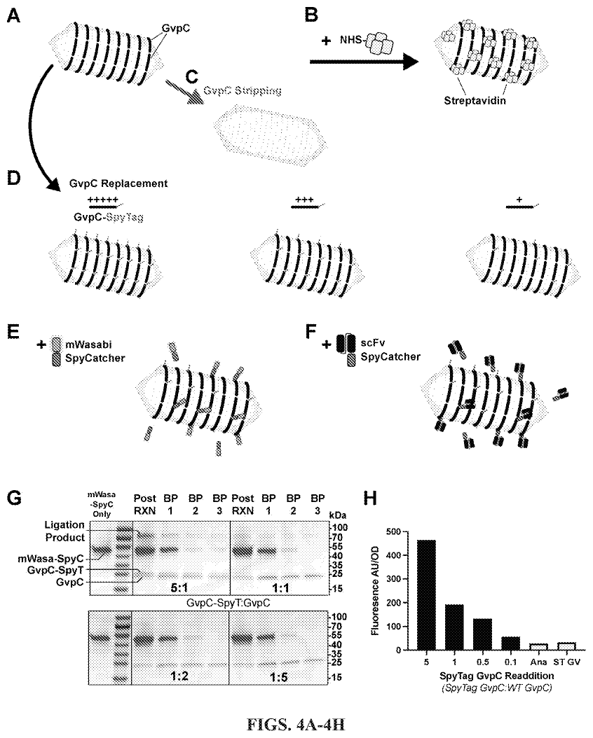

Modification of the shell protein as mentioned above may comprise addition of at least one affinity tag to a wildtype or recombinant shell protein, such as addition of an affinity tag at the C-terminus of gvpB (FIG. 1) or gvpC (FIG. 4). As used herein, the affinity tag is any moiety which binds tightly to a target with an affinity constant lower than 13×10⁻⁶ M. In some embodiments, the affinity tag binds to its target via a covalent bond. In other embodiments, the affinity tag binds to its target via a non-covalent bond. In some embodiments, the affinity tag may be a peptide. Specific examples of such peptide affinity tags known in the art include but are not limited to SnoopTag, SnoopTagJr, SnoopCatcher (also known as monomeric streptavidin, or mSA), SpyTag, SpyCatcher, DogTag, DogCatcher, or Strep-tacin (also known as Strep-tag II). A GV with an attached affinity tag is termed herein as a labeled GV. Additional examples of and details related to affinity tags are provided in the section that follows.

GVs can be modified utilizing numerous labeling strategies. In an embodiment, amino acid residues, for example lysine residues, on the surface of GVs may be modified, such as for example by EDC/NHS reaction, to attach an affinity tag to the surface of the GVs. GVs may, for example, be surface modified to comprise either biotin or streptavidin. Biotin-GVs and streptavidin-GVs are thus each contemplated as embodiments of labeled GVs. Streptavidin-GVs may be according to the present methods to bind to broad catalogue of commercially available biotinylated targets, such as antibodies that have been biotinylated. In some embodiments, the labeled GVs may be generated using commercially available one-step NHS-ester modifying reagents (e.g. NHS-fluorophore, NHS-streptavidin). In some embodiments, methods of the present disclosure provide for the conjugation of an antibody or antibodies to the surface amino acid of the GVs through methods known to those of skill in the art. In such embodiments, the antibody or antibodies are the affinity tags, and the corresponding antigen is the target protein as described herein. A GV-antibody conjugate is, in some embodiments, a labeled GV of the present disclosure.

In some embodiments, the present methods provide improved production process efficiency and cost advantages over traditional bead or resin-based systems. A salient feature of GVs is their amenability to pre-translational modifications. By genetically encoding specific ligands or proteins directly onto the GV shell proteins, it is possible to directly express modified GVs in bacterial systems. This results in the co-expression and assembly of functional GVs in a single step, streamlining the production process. Such an approach eliminates the need for subsequent conjugation steps, which are typically required in bead or resin systems. Traditional methods often necessitate the chemical synthesis and modification of beads, followed by the separate expression and purification of the desired recombinant protein. Once expressed and purified, this protein must then be chemically conjugated to the bead, adding multiple layers of complexity and cost. In contrast, GVs, by virtue of their buoyancy, can be easily isolated in the buoyant fraction, simplifying purification, and eliminating the need for secondary resins or other downstream processes. This integrated bioprocessing workflow not only enhances process efficiency but also offers substantial cost savings by reducing the need for multiple raw materials and extensive purification steps.

In some embodiments, the methods of the present disclosure comprise adding multiple independently selected affinity tags to at least one protein of a GV (FIG. 2). Therefore, the present methods may be used in a multiplexed purification of more than one target protein. As a non-limiting example, in some embodiments GVs are doubly labeled with two distinct, independently selected affinity tags: one affinity tag for EpCAM found on low count circulating tumor cells (CTCs) and one affinity tag for T cells, such as CD3 antibody. The present disclosure thus provides methods for purification of tumor-reactive T cells according to methods of the present disclosure.

In some embodiments, the present methods may purify cells comprising a certain a combination of target proteins. In some embodiments, two or more distinct collections of independently labeled GVs may be prepared or obtained such that the affinity tag of one of the collections of labeled GVs binds to the affinity tag of at least one of the distinct other collections of labeled GVs.

In some embodiments, the methods of the present disclosure may optionally comprise expression or attachment of a moiety to the GV which serves a distinct purpose from binding to a peptide, protein, or cell of the sample mixture. In some embodiments, this moiety may be an antibody, such as CD28. The present disclosure thus provides methods, for example, of simultaneous purification and activation of T cells. The methods of the present disclosure therefore represent an improvement with regard to the cell manufacturing life cycle over methods known in the art, wherein such activation typically is performed in a post-processing step after initial sorting.

In some embodiments, GVs are used according to the current methods as a bioprocessing reagent for biologics, and cell or gene therapies. For example, Fc binding proteins (FcBP) can be fused to GV shell proteins for buoyancy-based purification of antibodies (Choe el al., 2016). Viral vector purification currently has significant attention on improving purification and clearance processes. GVs recognizing capsids or an associated tag can be used for isolation by buoyancy. Alternatively, biologically active proteins can be functionalized onto GVs, acting a nanoparticle vehicle. An advantage of processing buoyant particles is that when centrifuged, they quickly float while all other matter tends to pellet. Soluble impurities can be depleted through cycles of isolating the floating layer and resuspending in fresh buffer (Lakshmanan et al., 2017). The equipment necessary for cell separation would be a centrifuge with the GV reagent acting as a general purpose “bead” or resin that is amenable to multiple modes of modification. Therefore, the present methods provide for a simplified or more economically favorable method of bioprocessing biologics or cell or gene therapies.

The methods of the present disclosure involve the incubation of labeled GVs with a sample mixture. This sample mixture may comprise of bacterial or mammalian cells, lysed cell solutions, proteins, or peptides. The sample mixture may comprise peptides or proteins which comprise the target or target protein of the affinity tag of the labeled GVs. In some embodiments, the sample mixture may comprise a population of cells which express the target or target protein on the surface of the cells of the sample mixture. The peptides, proteins, and cells of the sample mixture may express the target or target protein natively. In other embodiments, a sub-population or the entirety of the peptides, proteins, and cells of the sample mixture may be modified to possess the target or target protein of the affinity tag of the GVs using the principles and techniques of biochemistry or molecular biology as applied by a person skilled in the art. Further modifications of the peptides, proteins, or cells of the sample mixture, including any target or target protein possessed by any of the above, to improve solubility are contemplated. A non-limiting example of such a modification is the addition of maltose-binding protein (MBP) to a target peptide added through principles and techniques as applied by person skilled in the art to a peptide, protein, or cell of the sample mixture.

In some embodiments, the population of peptides comprising the target or target protein of the affinity tag of the labeled GV may represent a majority of the population of the peptides of the sample mixture. In some embodiments, the population of proteins comprising the target or target protein of the affinity tag of the labeled GV may represent a majority of the population of the proteins of the sample mixture. In some embodiments, the population of cells comprising the target or target protein of the affinity tag of the labeled GV may represent a majority of the population of the cells of the sample mixture. In some embodiments, the population of peptides comprising the target or target protein of the affinity tag of the labeled GV may represent a minority of the population of the peptides of the sample mixture. In some embodiments, the population of proteins comprising the target or target protein of the affinity tag of the labeled GV may represent a minority of the population of the proteins of the sample mixture. In some embodiments, the population of cells comprising the target or target protein of the affinity tag of the labeled GV may represent a minority of the population of the cells of the sample mixture.

In the methods of the present disclosure, the population of peptides possessing the target or target protein of the affinity tag of the labeled GV may be about 10%. About 20%, about 30%, about 40%, about 50%, about 60%, about 70%, about 80%, about 90%, or any range derivable therein, of the total population of peptides in the sample mixture. In some embodiments, the population of proteins possessing the target or target protein of the affinity tag of the labeled GV may be about 10%. About 20%, about 30%, about 40%, about 50%, about 60%, about 70%, about 80%, about 90%, or any range derivable therein, of the total population of proteins in the sample mixture. In some embodiments, the population of cells possessing the target or target protein of the affinity tag of the labeled GV may be about 10%. About 20%, about 30%, about 40%, about 50%, about 60%, about 70%, about 80%, about 90%, or any range derivable therein, of the total population of cells in the sample mixture.

In some embodiments, the population of peptides possessing the target or target protein of the affinity tag of the labeled GV may be less than 0.1%-10% of the total population of peptides in the sample mixture. In some embodiments, the population of proteins possessing the target or target protein of the affinity tag of the labeled GV may be less than 10% of the total population of proteins in the sample mixture. In some embodiments, the population of cells possessing the target or target protein of the affinity tag of the labeled GV may be less than 10% of the total population of cells in the sample mixture.

The labeled GVs, according to the methods of the present disclosure, are incubated with a sample mixture comprising peptides, proteins, or cells possessing the target(s) or target protein(s) of the affinity tag(s) of the labeled GVs for a first time period such that the peptides, proteins, or cells of the sample mixture possessing the target(s) or target protein(s) of the affinity tag(s) of the labeled GVs bind to the labeled GVs. The present disclosure thus provides methods for the formation of a captured target protein-gas vesicle composition. In some embodiments, the first time period is less than an hour. In some embodiments, the incubation of the labeled GVs with the sample mixture takes place at about 21 °C or about room temperature. In some embodiments, the incubation described above may take place at about 4 °C. For some embodiments, the corresponding captured targets may be soluble proteins inside the cells that will be released upon cell lysis. For embodiments of the present disclosure wherein a target or target protein is on the surface of an intact cell, the corresponding captured target protein-gas vesicle composition may be equivalently or interchangeably known as a captured target cell-gas vesicle composition. For some embodiments, the methods of the present disclosure do not require isolation or purification of target proteins on the surface of a cell. For some embodiments, the captured targets are intact cells through internalization of GVs that can be either mediated through binding to the surface proteins or not relying on any binding to the proteins but only the natural endocytosis process of the cells.

In some embodiments, the captured target protein-gas vesicle composition may have a different density than the labeled GVs or the targets or target proteins of the affinity tag of the labeled GVs. In embodiments of the present disclosure wherein the targets or target proteins are on the surface of a cell, the density of the captured target protein-gas vesicle composition may have a different density than the uncaptured cell. Native cell density tends to range between 1.06 to 1.07 g/cm (Miltenyi et al., 1990; Wang et al., 2022; Wang et al., 2023; Frenea-Robin et al., 2022; Hu et al., 2016; Liou et al., 2015; Weil et al., 2017; Palani et al., 2023; Zipursky et al., 1976). In some embodiments, cell density is reduced by contact with, such as binding to, the labeled GVs. In some embodiments, cell density is reduced by about 0.005 g/cm³, about 0.01 g/cm³, about 0.015 g/cm³, about 0.02 g/cm³, about 0.025 g/cm³, about 0.03 g/cm³, about 0.035 g/cm³, about 0.04 g/cm³, or any range derivable therein due to binding with labeled GV. In some embodiments, cell density is reduced by about 0.02 g/cm³, about 0.025 g/cm³, about 0.03 g/cm³, about 0.035 g/cm³, about 0.04 g/cm³, or any range derivable therein due to binding with labeled GV. In some embodiments, cell density is reduced by about 0.04 g/cm³ due to binding with labeled GV. In some embodiments, cell density is reduced by more than 0.04 g/cm³ due to binding with labeled GV. In some embodiments, the reduction in cell density due to binding with labeled GVs according to the present methods facilitates the formation of buoyant and non-buoyant fractions when using prepared density medias. The use of any commercially available density media or any media that may be prepared with a particular density according to methods known in the art is contemplated by the methods disclosed herein. In some embodiments, the media has a density of about 1.005 g/mL, about 1.01 g/mL, about 1.02 g/mL, about 1.03 g/mL, about 1.04 g/mL, about 1.05 g/mL, about 1.06 g/mL, about 1.07 g/mL, about 1.08 g/mL, or any range derivable therein. In some embodiments, the present methods involve the use of media with a density of about 1.04 g/mL. In other embodiments, the present methods involve the use of media with a density of about 1.06 g/mL.

The methods described herein provide for a second time period, wherein the captured target protein-gas vesicles may separate within the sample mixture according to density. In some embodiments, the second time period may be concurrent with the first time period described above. In some embodiments, the second time period may overlap with the first time period. In other embodiments, the second time period may follow the first time period.

In some embodiments, the sample mixture is left undisturbed during the second time period. In some embodiments, the sample mixture is left undisturbed during the second time period for at least 15 minutes. In some embodiments, the sample mixture may be left undisturbed during the second time period for about 1 hour, about 1.5 hours, about 2 hours, about 2.5 hours, about 3 hours, about 3.5 hours, about 4 hours, about 4.5 hours, about 5 hours, about 5.5 hours, about 6 hours, about 6.5 hours, about 7 hours, about 7.5 hours, about 8 hours, about 8.5 hours, about 9 hours, about 9.5 hours, about 10 hours, or any range derivable therein. In some embodiments, the second time period is about 4 hours to about 6 hours. In some embodiments, the second time period is about 5 hours. The sample mixture may be left undisturbed during the second time period overnight, or for any amount of time until the particle has been observed to have accumulated at the sample mixture-air interface. In some embodiments, particle accumulation at the sample mixture-air interface is indicated by the formation of a white layer of gas vesicles.

The second time period may comprise a centrifugation-assisted flotation step (FIG. 3). The sample mixture captured target protein-gas vesicle composition may be centrifuged at, for example, 400×g. The sample mixture comprising the captured target cell-gas vesicle compositions may be centrifuged for about 5 minutes, about 10 minutes, about 15 minutes, about 20 minutes, about 25 minutes, about 30 minutes, about 35 minutes, about 40 minutes, about 45 minutes, about 50 minutes, about 55 minutes, about 60 minutes, or any range derivable therein. The second time period may comprise more than one centrifugation-assisted flotation steps. In some embodiments, the methods of the present disclosure comprise one, two, three, four, or five centrifugations.

In some embodiments, the methods disclosed herein optionally comprise density-gradient centrifugation or counterflow elutriation of the sample mixture containing the captured target protein-gas vesicle. Elutriation is a counterflow-centrifugal device that separates cells based off size and density, balance the g-force and a counter-acting drag-force introduced from fluid flow (Coulais et al., 2012; Stroncek et al., 2014). While it is effective at separating peripheral blood monocular cells (PBMCs) from red blood cells, it cannot separate lymphocytes from one another due to their similar size and density. GVs displaying labels, for example, antibody fragments, that recognize specific cell-surface markers may selectively associate with cells, effectively reducing their density for separation using counter-flow centrifugal forces as a more continuous process than using discrete batches of density media.

In some embodiments, the captured target protein-gas vesicle compositions redistribute within the sample mixture during the second time period to accumulate at the sample mixture-air interface. In some embodiments, the captured target protein-gas vesicle compositions redistribute within the sample mixture during the second time period but do not reach the sample mixture-air interface.

Any of the affinity tags of the labeled GVs or the corresponding binding sites of the affinity tags found on the target, target protein, or cell which has the target protein or target peptide on the surface of the cell, may in some embodiments possess a protease cleavage site or a self-splicing peptide sequence. Protease cleavage sites or self-splicing sequences are contemplated by the present methods for both native recognition sites and for recognition sites that are installed on targets, target proteins, or cells which have the target protein or target peptide on the surface of the cell by a person of skill in the art. A non-limiting example of a protease cleavage site contemplated by the present disclosure is a TEV protease cleavage site. A non-limiting example of a self-splicing peptide sequence contemplated by the methods of the present disclosure is an intein. The methods of the present disclosure optionally comprise protease mediated cleavage or self-splicing at the protease cleavage site or at the self-splicing sequence, respectively, to separate the target protein from the captured target protein-gas vesicle composition. In some embodiments, affinity tag modification of GVs may not be required for internalization of GVs by the target cells. In some embodiments, chemical conjugation of antibodies to GVs will be used that will not be developed together with the self-splicing peptide sequence. In some embodiments, the self-splicing peptide allows for increased purity of the downstream purified target protein of interest. According to the present methods, the presence or inclusion of a self-splicing sequence within any component of the labeled GV does not imply or require that self-splicing of the sequence will occur. In some embodiments, the captured target protein-gas vesicle composition or the captured target cell-gas vesicle composition, will be isolated intact and without separation of the target protein or target cell from the gas vesicle.

### II. AFFINITY TAGS

The present disclosure provides labeled GVs, which comprise an affinity tag that in some embodiments binds to a target located on the surface of a cell. In certain embodiments, the affinity tag is a peptide sequence that is engineered according to the present disclosure. Modification of the GV surface as described in the section above above may comprise addition of at least one affinity tag to the shell protein, such as addition of an affinity tag at the C-terminus of gvpB (FIG. 1). Affinity tags, according to the embodiments may be, for example, an antibody, a lipid, a carbohydrate, a polysaccharide, a growth factor, a hormone, a peptide, an aptamer, a small molecule such as a hormone, an imaging agent, or cofactor, or a cytokine. In some embodiments, the affinity tag is a functional group that associates with the cell membrane, a carbohydrate or polysaccharide that binds to one or more markers on the cell membrane, a lipid that binds to one or more markers on the cell membrane, a small molecule that binds to one or more markers on the cell membrane, an aptamer that binds to one or more markers on the cell membrane, or a peptide or an antibody that binds to one or more markers on the cell membrane. In some embodiments, the affinity tag may target a human cell, such as a cancer cell or a T cell. For instance, an affinity tag according to the embodiments may bind to a low count circulating tumor cell (CTC). It has been demonstrated that the gp240 antigen is expressed in a variety of melanomas but not in normal tissues. Thus, in some embodiments, the compositions of the present disclosure may be used in conjugates with an antibody for a specific antigen that is expressed by a cancer cell but not in normal tissues.

In certain embodiments, the affinity tag is a functional group such as a positively charged group like an amine. The positively charged group may be used to associate with the negatively charged groups at the surface of the cell membrane. It is contemplated that this group might be used to associate with other negatively charged groups such as negatively charged proteins or nucleic acids.

In certain additional embodiments, it is envisioned that affinity tags bind to multiple types of cancer cells. For example, the 8H9 monoclonal antibody and the single chain antibodies derived therefrom bind to a glycoprotein that is expressed on breast cancers, sarcomas and neuroblastomas (Onda, et al., 2004). Another example is the cell targeting agents described in U.S. Patent Publication No. 2004/005647 and in Winthrop, et al. (2003) that bind to MUC-1, an antigen that is expressed on a variety cancer types. Thus, it will be understood that in certain embodiments, affinity tags according the embodiments may be targeted against a plurality of cancer or tumor types.

Additionally, certain cell surface molecules are highly expressed in tumor cells, including hormone receptors such as human chorionic gonadotropin receptor and gonadotropin releasing hormone receptor (Nechushtan et al., 1997). Therefore, the corresponding hormones may be used as the affinity tags in cancer therapy. Additionally, the affinity tag that may be used include a cofactor, a sugar, a drug molecule, an imaging agent, or a fluorescent dye. Many cancerous cells are known to over express folate receptors and thus folic acid or other folate derivatives may be used as affinity tags to allow for specific binding of labeled GVs of the present disclosure cell (Campbell, et al., 1991; Weitman, et al., 1992).

Since a large number of cell surface receptors have been identified in hematopoietic cells of various lineages, ligands or antibodies specific for these receptors may be used as affinity tags. IL-2 may also be used as an affinity tag in a chimeric protein to target IL-2R+ cells. Alternatively, other molecules such as B7-1, B7-2 and CD40 may be used to specifically target activated T cells (The Leucocyte Antigen Facts Book, 1993, Barclay, et al. (eds.), Academic Press). Furthermore, B cells express CD19, CD40 and IL-4 receptor and may be targeted by affinity tags that bind these receptors, such as CD40 ligand, IL-4, IL-5, IL-6 and CD28. The elimination of immune cells such as T cells and B cells is particularly useful in the treatment of lymphoid tumors.

Other cytokines that may be used to target specific cell subsets include the interleukins (IL-1 through IL-15), granulocyte-colony stimulating factor, macrophage-colony stimulating factor, granulocyte-macrophage colony stimulating factor, leukemia inhibitory factor, tumor necrosis factor, transforming growth factor, epidermal growth factor, insulin-like growth factors, and/or fibroblast growth factor (Thompson (ed.), 1994, The Cytokine Handbook, Academic Press, San Diego). In some aspects, the targeting peptide is a cytokine that binds to the Fn14 receptor, such as TWEAK (see, e.g., Winkles, 2008, incorporated herein by reference).

A skilled artisan recognizes that there are a variety of known cytokines, including hematopoietins (four-helix bundles) [such as EPO (erythropoietin), IL-2 (T-cell growth factor), IL-3 (multicolony CSF), IL-4 (BCGF-1, BSF-1), IL-5 (BCGF-2), IL-6 IL-4 (IFN-b2, BSF-2, BCDF), IL-7, IL-8, IL-9, IL-1l, IL-13 (P600), G-CSF, IL-15 (T-cell growth factor), GM-CSF (granulocyte macrophage colony stimulating factor), OSM (OM, oncostatin M), and LIF (leukemia inhibitory factor)]; interferons [such as IFN-g, IFN-a, and IFN-b); immunoglobin superfamily (such as B7.1 (CD80), and B7.2 (B70, CD86)]; TNF family [such as TNF-a (cachectin), TNF-b (lymphotoxin, LT, LT-a), LT-b, CD40 ligand (CD40 L), Fas ligand (FasL), CD27 ligand (CD27 L), CD30 ligand (CD30 L), and 4-1BBL)]; and those unassigned to a particular family [such as TGF-b, IL 1a, IL-1b, IL-1 RA, IL-10 (cytokine synthesis inhibitor F), IL-12 (NK cell stimulatory factor), MIF, IL-16, IL-17 (mCTLA-8), and/or IL-18 (IGIF, interferon-g inducing factor)]. Furthermore, the Fc portion of the heavy chain of an antibody may be used to target Fc receptor-expressing cells such as the use of the Fc portion of an IgE antibody to target mast cells and basophils.

Furthermore, in some aspects, the affinity tag is a peptide sequence, as described above, or a cyclic peptide. Examples of cell- and tissue-targeting peptides that may be used according to the embodiments are provided, for instance, in U.S. Pat. Nos. 6,232,287; 6,528,481; 7,452,964; 7,671,010; 7,781,565; 8,507,445; and 8,450,278, each of which is incorporated herein by reference.

Thus, in some embodiments, affinity tags are antibodies or avimers. Antibodies and avimers can be generated against virtually any cell surface marker thus, providing a method for targeted binding of a labeled GV to virtually any cell population of interest. Methods for generating antibodies that may be used as affinity tags are known in the art and are detailed below. Methods for generating avimers that bind to a given cell surface marker are detailed in U.S. Patent Publications Nos. 2006/0234299 and 2006/0223114, each incorporated herein by reference. Any other antigen-specific biomolecule, non-limiting examples of which include nanobodies and aptamers, is an affinity tag in certain embodiments of the present disclosure.

Additionally, it is contemplated that the compositions described herein may be comprise a nanoparticle or other nanomaterial. Some non-limiting examples of nanoparticles include metal nanoparticles such as gold or silver nanoparticles or polymeric nanoparticles such as poly-L-lactic acid or poly(ethylene) glycol polymers. Nanoparticles and nanomaterials which may be conjugated to the instant compounds include those described in U.S. Patent Publications Nos. 2006/0034925, 2006/0115537, 2007/0148095, 2012/0141550, 2013/0138032, and 2014/0024610 and PCT Publication No. 2008/121949, 2011/053435, and 2014/087413, each incorporated herein by reference.

### III. KITS

The technology disclosed herein includes kits for forming labeled GVs for purifying peptides, proteins, or cells. In some embodiments, the kits described herein may comprise pre-made labeled GVs for purifying peptides, proteins, or cells. A “kit” refers to a combination of physical elements. For example, a kit may include, for example, one or more components, such as specific primers, enzymes, reaction buffers, an instruction sheet, and other elements useful to practice the technology described herein. These physical elements can be arranged in any way suitable for carrying out the disclosure.

The components of the kits may be packaged either in aqueous media or in lyophilized form. The container means of the kits will generally include at least one vial, test tube, flask, bottle, syringe or other container means, into which a component may be placed, and preferably, suitably aliquoted (e.g., aliquoted into the wells of a microtiter plate). Where there is more than one component in the kit, the kit also will generally contain a second, third or other additional container into which the additional components may be separately placed. However, various combinations of components may be comprised in a single vial. The kits of the present disclosure also will typically include a means for containing the labeled GVs, and any other reagent containers in close confinement for commercial sale. Such containers may include injection or blow molded plastic containers into which the desired vials are retained.

A kit will also include instructions for employing the kit components as well the use of any other reagent not included in the kit. Instructions may include variations that can be implemented. It is contemplated that such reagents are embodiments of kits of the disclosure. Such kits, however, are not limited to the particular items identified above.

### IV. PROCESS SCALE-UP

The methods described herein can be further modified and optimized for preparative, pilot- or large-scale production, either batch of continuous, using the principles and techniques of process chemistry, bacterial growth, or bioprocessing as applied by a person skilled in the art. Such principles and techniques are taught, for example, in Practical Process Research & Development (2000), which is incorporated by reference herein. The methods described herein may be used to produce preparative scale amounts of any of the compounds or compositions described herein.

### V. DEFINITIONS

The use of the word “a” or “an,” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the inherent variation of error for the device, the method being employed to determine the value, or the variation that exists among the study subjects or patients. Unless otherwise noted, the term “about” is used to indicate a value of ±100% of the reported value, preferably a value of ±5% of the reported value. It is to be understood that, whenever the term “about” is used, a specific reference to the exact numerical value indicated is also included.”

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and also covers other unlisted steps.

The above definitions supersede any conflicting definition in any reference that is incorporated by reference herein. The fact that certain terms are defined, however, should not be considered as indicative that any term that is undefined is indefinite. Rather, all terms used are believed to describe the invention in terms such that one of ordinary skill can appreciate the scope and practice the present invention.

### VI. EXAMPLES

The following examples are included to demonstrate preferred embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### A. Cell Sorting

a. SpyTag GVs Allow Modular Functionalization with SpyCatcher Fusion Proteins

In some embodiments, the body of gas vesicles used in the present methods is composed of repeating subunits of major shell protein GvpA, which can be reinforced at the aqueous boundary by GvpC (FIG. 4A). GVs can, in some embodiments, be simply modified using known conjugation techniques, for example NHS-ester chemical modification reagents to conjugate to lysine residues or N-terminal free primary amines (FIG. 4B). Through a more structured protein engineering approach, GvpC minor shell proteins can be stripped (FIG. 4C) or replaced with engineered variants. In some embodiments, SpyTag3 (hereon referred to as SpyTag) was fused to the C-terminus of GvpC so that it can be displayed on the outer surface of the GV body (FIG. 4D) for modular functionalization with SpyCatcher fusions such as mWasabi-SpyCatcher or anti-CD3 scFv-SpyCatcher (FIG. 4E, FIG. 4F). Incubation of SpyTag GVs with SpyCatcher-mWasabi fusion resulted in modification of GVs with the fluorescent protein that is detectable by the formation of a ligation product between SpyTag and SpyCatcher that appears as a higher MW band on SDS-Page (FIG. 4G). Ligation of mWasabi-SpyCatcher also resulted in an enrichment of fluorescent signature in the buoyant fraction containing GVs (FIG. 4H). Furthermore, the concentration of mWasabi-SpyCatcher on GVs can be adjusted by controlling the amount of GvpC-SpyTag input during GvpC replacement on GVs.

b. Gas Vesicles Reduce Cell Density for Density-Based Isolation

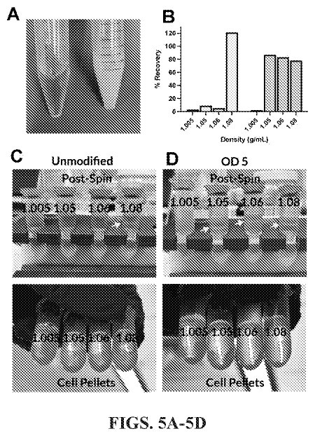

Initially, unmodified WT GVs were tested with RAW 264.7 macrophages by incubating them together and measured a maximum of 0.03 g/cm³ reduction in cell density (FIG. 5). Density-based cell separation strategies relying on phagocytosis require extended incubation times at 37 °C for robust GV internalization.

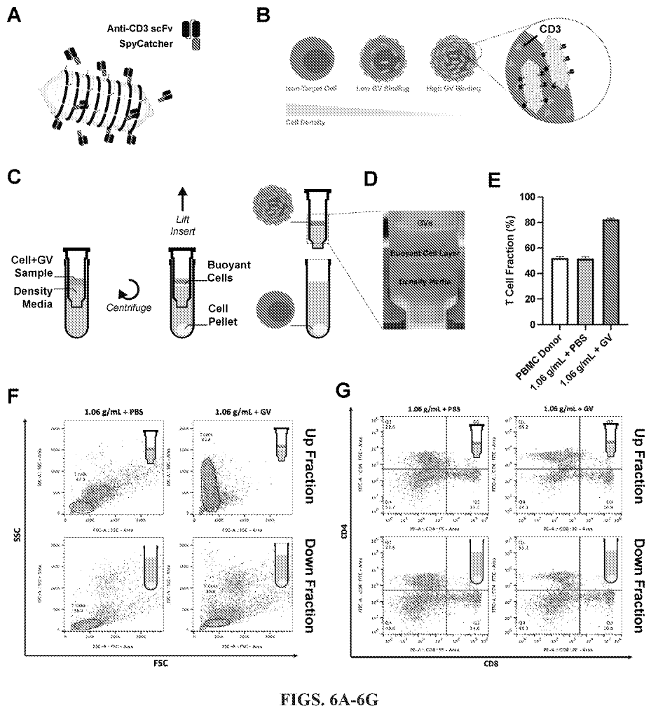

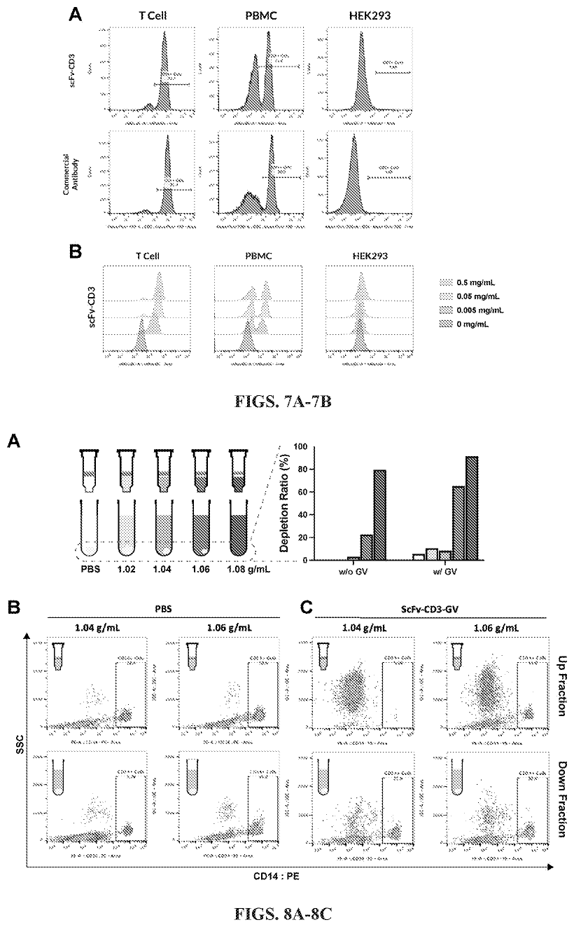

GVs for antigen-specific sorting, such as for sorting T-cells, were developed by attaching an anti-CD3 single chain variable fragment (scFv) to GvpC using the SpyTag/SpyCatcher ligation system (FIG. 6A). Prior to GV modifications, the reference CD3-scFv sequence was first validated with cell-binding studies. CD3 scFv-mWasabi was cloned and expressed in E. coli. Purified CD3 scFv-mWasabi was applied to HEK cells (negative), a pure patient-derived T-Cells sample (positive), and freshly isolated peripheral blood monocular cells (PBMCs; mixed sample), and it showed staining distributions comparable to commercial anti-CD3 antibody (OKT3 clone) (FIG. 7). CD3 scFv with a C-terminal SpyCatcher fusion was then cloned, expressed, and purified prior to ligation with SpyTag GVs. By increasing total binding of GVs, cell density can be proportionately reduced on CD3+ T-Cells (FIG. 6B). Thus, in some embodiments the present invention provides methods with improved predictability and control for cell sorting compared to methods known in the art. For example, a common preparation of PBMCs from a blood involves overlaying a buffy coat over Ficoll-Paque, a media with a specific gravity of 1.08 g/mL (Turner et al., 2020). While PBMCs may be captured as a buoyant cell layer above the dense Ficoll media, the even more dense red blood cells will form a distinct fraction below the Ficoll at the bottom of the vessel. Beyond isolating PBMCs, density media alone has limited ability to sort for specific cell types, such as T-Cells. According to the methods disclosed herein, GVs bind to specific cell surface markers which can then be isolated using existing and relatively cost-effective density medias.

One of the initial challenges when working with buoyant samples was efficient and complete isolation of the floating fraction. To overcome this obstacle, a microtube spin column was repurposed to quickly separate the buoyant top fraction and the pelleting bottom fraction (FIG. 6C). Rather than using a filter or packed beads within the column, it was left open and filled with media modified to specific densities. Cells with an effective density that is lower than modified media were retained in the top buoyant fraction after centrifugation, which can be completely and easily isolated by removing the column insert and transferring to a secondary vessel. This system provides a device-based solution for isolating buoyant fractions that are otherwise easy to disturb and tedious to fully recover.

CD3-GVs were incubated with freshly isolated PBMCs and fractionated over 1.06 g/mL to evaluate cell capture in the buoyant fraction. A robust increase in SSC signal was observed in the CD3-GV treated sample, particularly in the top, non-pelleting fraction. Significant increases in SSC indicate, without being bound by theory, higher degrees of GV binding. Gating for CD4+ and CD8+ cells in each of the fractions, the greatest enrichment of T-Cells was observed in the top fraction of PBMCs treated with CD3-GVs: over 80% of the isolated sample was composed of T-Cells compared to 50% of both the original input and the top fraction of the PBS-treated control. Based on FSC and SSC profile of a secondary group of cells, the presence of a sub-population with both high FSC and SSC indicate monocyte contamination when samples are isolated over 1.06 g/mL density media.

c. Density Media Composition Reduces Monocyte Contamination

To reduce monocyte contamination, the density for the media used for sorting was decreased. CD3-GVs were applied to freshly isolated PBMCs and separated across a serial dilution of Ficoll-Paque ranging in density from 1.005 g/mL (PBS) to 1.08 g/mL with a 0.02 g/mL step size (FIG. 8A). At the upper range of density, both PBS and CD3-GV treated samples showed near total depletion of a pelleting fraction at 1.08 g/mL because it was equivalent to the density originally used for PBMC isolation. Differences in the buoyant fraction cell count can first be observed at 1.06 g/mL where 20% of the original cell input no longer pelleted when treated with PBS, but 60% of the cells no longer pelleted when treated with CD3-GVs. Further decreasing density to 1.04 g/mL reliable captures a buoyant cell fraction when samples are treated with GVs, although at a much lower total yield compared to 1.06 g/mL. Also, cell count events were only observed in the top fraction of 1.02 g/mL and 1.005 g/mL samples when treated with CD3-GVs, not when only treated with PBS.

The % distribution of monocyte in the separated fractions was measured using flow cytometry. PBMCs not treated with CD3-GVs showed a % monocyte population of 50.6% over 1.06 g/mL density media, and 18.4% over 1.04 g/mL density media (FIG. 8B). In comparison, reducing the density media from 1.06 g/mL to 1.04 g/mL for PBMCs treated with CD3-GVs resulted in a depletion of monocytes from the top fraction from 11% to 0.18% (FIG. 8C). A high SSC signature in the top fraction of GV-treated cells continued to be consistent with GV-Cell interaction, driving density reduction of target T-cells. While reducing the density media improves the final purity of the isolated T-cells, it corresponds with reduced overall total cell yield. It is plausible, without being bound by theory, that only the cells with a high density of surface antigens available for GV binding achieve greater reductions in cell density.

d. Streptavidin-Modified GVs can be Combined with Commercial Biotinylated Antibodies

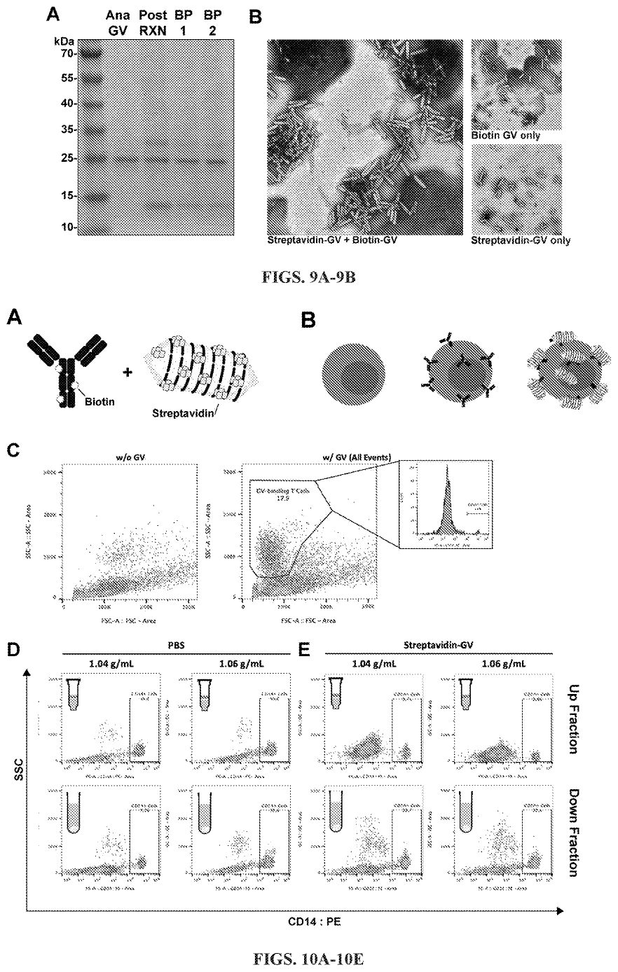

The present methods may also involve the use of streptavidin-modified GVs with commercially available antibodies that are biotinylated. Lysine residues and termini on GV shell proteins were targeted with an NHS-Streptavidin modification kit and ligation was confirmed by SDS-Page (FIG. 9A). After chemical reaction and multiple buoyancy purifications, the retention of streptavidin monomer and multimer bands in the buoyant fraction indicated, without being bound by theory, successful modification of GVs. The functionality of streptavidin GVs was further confirmed in clustering assays where they were mixed with biotinylated GVs and GV-GV interaction behavior was observed using TEM-imaging (FIG. 9B).

Functionally validated streptavidin-GVs were used in a 2-part cell labeling process where the sample was first stained with biotinylated CD3 antibody (Clone: OKT3) followed by secondary labeling with streptavidin-GVs (FIG. 10A, FIG. 10B). A biotin-streptavidin based system for GV cell labeling resulted in increases in SSC within a sub-population of the PBMC sample, similarly to scFv-GVs assembled using the Spy system (FIG. 10C). Untreated PBMCs demonstrated a 50.6% CD14+ monocyte load in the top fraction over 1.06 g/mL density media, and 18.4% monocyte load over 1.04 g/mL (FIG. 10D). Comparatively, treatment of PBMCs with anti-CD3 biotinylated antibody and Streptavidin-GVs showed only 6.9% monocytes burden over 1.06 g/mL media and 8.71% monocytes over 1.04 g/mL. The remaining fraction is presumably positively isolated CD3+ T-Cells, based off previous CD4+/CD8+ assessment, FSC signature of the isolated cells, and low-density media concentration used.

e. Methods

### i Gas Vesicle Harvesting and Buoyancy Purification

Gas vesicles (GVs), as mentioned above, are produced natively in Anabaena flos-aquae or Haloarchae salinarum, or heterologously in Escherichia coli according to procedures described in prior publications. Anabaena flos-aquae was cultivated for the native cyanobacteria expression of GVs. Anabaena flos-aquae cultures were inoculated in BG11 freshwater media and incubated in a 1% CO2 incubator modified with LED strip lighting (2000 lumens) on a 12 Hr on/off light/dark cycle. Anabaena flos-aquae was cultivated for approximately a month before GV harvesting. GV harvesting was performed by first allowing the Anabaena flos-aquae to float in a separatory funnel before draining the clarified subnatant. Concentrated Anabaena flos-aquae were lysed by mixing with an equal volume of 2× homemade lysis buffer (1M Sorbitol, 100 mM Tris-HCl, and 1 wt % Tween-80) and rocking overnight at 4 °C. Complete lysis was indicated by optical transparency of the solution (i.e. no particulates). The lysis buffer was removed by first centrifuging the lysis solution at 200 rcf for 24 hours at 4 °C. The released gas vesicles concentrated at the air-water interface and formed a white film on the surface. After lysis, released GVs were washed through multiple rounds of buoyancy purification where each round involved overnight centrifugation (200×g, 4 °C) followed by removal of the subnatant using a syringe submerged under the floating GV layer. The dewatered GV sample was then resuspended in fresh TBS and the buoyancy purification cycle was repeated. GVs underwent a final concentration cycle to a stock OD₅₀₀=30 (~1 mg/mL of protein in PBS). The number of cycles required was dependent percentage of subnatant removed and dilution factor of each wash. At least a 1000×DF was found to be necessary for complete depletion of soluble impurities.

### ii Cloning

Plasmids for the recombinant expression of GvpC-SpyTag and SpyCatcher fusions were constructed using Gibson assembly of amplified PCR products. PCR of inserts and the pET28a vector was performed using amplification primers with designed overhangs for assembly using Gibson Assembly Master Mix (New England Biolabs). Recombinant GvpC was designed with a C-terminal SpyTag followed by C-terminal his-tag. SpyCatcher fusions were designed to include a C-terminal his-tag and an N-terminal fusion to a protein of interest, either mWasabi or anti-CD3 scFv. Plasmid assembly transformed into Turbo Competent E. coli, culturing overnight in LB containing 50 μg/mL Kanamycin, and plasmids were isolating using the QIAprep® Miniprep plasmid DNA extraction kit for sequencing validation through Sanger sequencing using a T7 promoter and T7 terminator forward and reverse sequencing primers.

In some embodiments, the major shell protein gvpB was modified by adding to the C-terminus with the amino acid sequence GSGKLGDIEFIKVNK (SEQ ID NO:1). Note that KLGDIEFIKVNK (SEQ ID NO:2) is the previously described sequence of the SnoopTag (Veggiani et al, 2016). In a separate experiment, the major shell protein gvpB was modified by adding to the C-terminus with the amino acid sequence GSGWSHPQFEK (SEQ ID NO:3), where WSHPQFEK (SEQ ID NO:4) is the previously described sequence of the StrepTag II (Korndofer, 2002). The constructed plasmid was transformed into BL21 Star™ (DE3)pLysS One Shot™ Chemically Competent E. coli. The culture was induced with 20 μM IPTG at OD₆₀₀=0.4 and incubated overnight at 30 °C for 22 hours. GVs were harvested by adding SoluLyse™ Bacterial Protein Extraction Reagent and Dnase I, and purified by repeating buoyancy purification via low-speed centrifugation (400×g) at 4 °C and 1×PBS washing. Purified high-density surface tagged GVs were stored at 4 °C.

In some embodiments, the surface of GVs may be modified through chemical conjugation through various click chemistry kits, engineered peptides specific to antibodies, or other techniques known in the art.

### iii Recombinant Protein Expression and Purification

Plasmids were heatshock transformed into either Shuffle T7 Express for plasmids expressing of anti-CD3 scFv fusion proteins or BL21(DE3) chemically competent cells for all other protein expressions. After overnight culture with Kanamycin antibiotic selection (50 ug/mL), 100 mL of fresh LB containing Kanamycin was inoculated to an OD600=0.05 and incubated until an OD600=0.4-0.5 was reached, at which point protein expression was induced using 400 mM IPTG followed by expression for 20 hours and 30 °C. Cells were harvested by centrifuge at 2000×g for 20 minutes at 4 °C and then resuspended in Solulyse lysis reagent supplemented with Lysozyme (250 ug/mL), DnaseI (10 ug/mL), and MgCl2 (2 mM). To further promote lysis, cell suspension was sonicated with 5 rounds of 20 seconds on/off cycles on ice using a Branson Sonifier 250 set at an output control of 6. For soluble proteins, the lysed cells were centrifuged at 20,000×g for 20 minutes at 4 °C to pellet the insoluble fraction. The soluble supernatant was incubated with Ni-NTA Agarose beads that were pre-equilibrated with wash buffer (20 mM Tris, 500 mM NaCl, 20 mM Imidazole). After 1 Hr, flow-through was collected and beads were washed with wash buffer before elution using elution buffer (20 mM Tris, 500 mM NaCl, 250 mM Imidazole) (FIG. 11B, FIG. 11C). For GvpC purification, the lysed cell solution was centrifuged at 20,000×g for 20 minutes at 4 °C. The soluble supernatant was discarded, and the pellet was resuspended in wash buffer supplemented with 6M urea. After rocking for 30 minutes at 4 °C, the solution was centrifuged at 20,000×g for 20 minutes at 4 °C. The supernatant was transferred to incubate with Ni-NTA agarose beads pre-equilibrated in a wash buffer supplemented with 6M urea. Elution buffer was also supplemented with 6M urea (FIG. 11A). For all protein purifications, elutions were pooled and concentrated in a 10,000 kDa MWCO Amicon filter insert, and buffer exchanged to remove imidazole through three rounds of concentration and resuspension with fresh TBS for soluble proteins or TBS with 6M urea for GvpC-SpyTag.

In some embodiments, soluble protein of interest (POI) was fused on the C-terminus of catcher proteins, SnoopCatcher/monomeric streptavidin (mSA), which is described in (Keeble et al, 2019; DeMonte et al., 2013). In a bench-scale trial, Superfolder GFP (sfGFP) was applied as POI. The constructed plasmid was transformed into BL21 Starm (DE3)pLysS One Shot™ Chemically Competent E. coli. The culture was induced with 400 μM IPTG at OD600=0.8 and incubated overnight at 18 °C for 20 hours. The culture was then centrifuged at 3000×g for 30 minutes to remove the liquid medium, and the cells were resuspended in prepared lysis buffer. Cells in lysis buffer were quickly frozen in liquid nitrogen and thawed in a 42 °C water bath, repeating the freeze-thaw five times. The cell lysis was then sonicated 10 seconds for five times. Dnase I, activated by adding MgCl₂, was added to cell lysis and incubated at room temperature for 30 minutes. The cell lysate was spun down using 20000×g for 20 minutes to separate supernatant and pellet.

### iv Protein Quantification

Concentration of purified and buffer exchanged proteins was determined using the Bradford reagent. 1 mg/mL BSA standard was prepared in either 1×TBS (pH 7.4) to determine soluble protein concentration or in 6M Urea in 1×TBS to determine the concentration of recombinant GvpC. 5 μL of sample or standard was mixed with 250 μL of Bradford reagent before mixing and reading absorbance at the 595 nm wavelength. A standard curve of BSA was prepared from 0-1 mg/mL and compared to diluted protein samples to extrapolate original unknown sample protein concentration.

In some embodiments, SnoopTag tagged GVs were incubated with lysis supernatant of Snoopcatcher-POI for a given time to allow for the binding. For example, for SnoopCatcher-sfGFP, 2 hours at 4 °C allowed efficient binding. Next, the buoyancy-assisted protein purification proceeded with either gravity flotation, or with centrifugation-assisted flotation. For gravity flotation, the solution was left undisturbed for 5.3 hours. Theoretical estimation based on the hydrodynamic radius of the GV complex (~1402.2 nm) showed that the particles should rise by ~4E-5 mm every hour. Gravity flotation protocols resulted in particle accumulation at the top surface of 2 cm height water after 2 hours. For centrifugation-assisted flotation, the solution was centrifuged at 400×g for 15 minutes, and the sub-natant was removed by syringe. The remaining top fraction of GV-POI was resuspended with 1×PBS. The centrifugation and resuspension process was repeated three times to improve the purity. The enrichment of GV-POI in each repeated centrifugation run was calculated as the ratio of the remaining top fraction to the total volume of the solution. The purity of an sfGFP sample was assessed by fluorescent intensity to indicate the POI (sfGFP) bound on GVs, and the results showed 81.9, 99.3, 99.6% purity after 3 repeats.

### v GvpC Replacement on WT GV

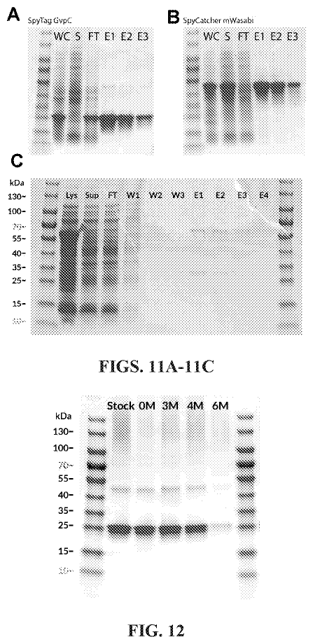

Recombinant GvpC was replaced on WT GV through solubilization of native GvpC and re-annealing of GvpC-SpyTag. Increasing molar ratios of GvpC-SpyTag relative to the WT GvpC present on GVs (0.1:1-5:1. GvpC-SpyTag:WT GvpC) was reconstituted with WT GVs in the presence of 6M urea. 6M urea effectively solubilizes GvpC and its derivates from the primary GV body (FIG. 12), and rapidly diluting the sample with TBS to a final 3M urea resulted in re-annealing of GvpC and GvpC-SpyTag GVs. Excess and unannealed GvpC, as well as residual urea can be removed through two rounds of buoyancy purification.

### vi SpyTag-SpyCatcher Ligation Assay and Fluorescent Quantification

SpyTag modified GVs resuspended in 1×TBS (pH 7.4) were incubated with SpyCatcher fusions and incubated together overnight at 4 °C. During ligation reactions, SpyTag-GvpC was present at approximately 1 uM and SpyCatcher fusions were added at a 5-20 fold molar excess. Excess and unreacted SpyCatchers were removed through multiple rounds of buoyancy purification. After buoyancy purification, the relative amount of mWasabi-SpyCatcher ligated to prepared GVs was determined by fluorescent intensity measurement using 485/529 nm (ex/em) wavelengths and automatically adjusted gain and normalized to the final OD of processed GV samples.

In some embodiments, SnoopTag tagged GVs were incubated with lysis supernatant of Snoopcatcher-POI for a given time to allow for binding. For example, for SnoopCatcher-sfGFP, 2 hours at 4 °C allowed efficient binding. Next, the buoyancy-assisted protein purification proceeded with either gravity flotation, or with centrifugation-assisted flotation. For gravity flotation, the solution was left undisturbed for 5.3 hours. Theoretical estimation based on the hydrodynamic radius of the GV complex (~1402.2 nm) showed that the particles should rise by ~4E-5 mm every hour. Gravity flotation protocols resulted in particle accumulation at the top surface of 2 cm height water after 2 hours. For centrifugation-assisted flotation, the solution was centrifuged at 400×g for 15 minutes, and the sub-natant was removed by syringe. The remaining top fraction of GV-POI was resuspended with 1×PBS. The centrifugation and resuspension process was repeated three times to improve the purity. The enrichment of GV-POI in each repeated centrifugation run was calculated as the ratio of the remaining top fraction to the total volume of the solution. The purity of an sfGFP sample was assessed by fluorescent intensity to indicate the POI (sfGFP) bound on GVs, and the results showed 81.9, 99.3, 99.6% purity after 3 repeats.

### vii SDS-Page

SDS-Page was performed using 4-15% Mini-PROTEAN TGX Stain-Free Protein Gels. Samples were mixed with an equal volume of 2× Laemmli sample buffer containing 2-mercaptoethanol as a reducing agent. Gels were run at 100V using a running buffer composed of 25 mM Tris, 192 mM glycine, 0.1% SDS at pH 8.3. Run gels were stained with Coomassie brilliant blue solution before destaining and imaging. Gels were observed for the presence of a larger MW ligation band indicating successful covalent linking of SpyTag to SpyCatcher fusion proteins.

viii NHS-Modification of GVs

NHS modifications were accomplished using Lightning Link chemical modification kits according the supplier instructions for both biotin and streptavidin modification of GVs. Purified and concentrated GVs (OD₅₀₀=50) were added directly to the lyophilized NHS modification reagent and allowed to react overnight at 4 °C. Unreacted and freely soluble biotin or streptavidin was depleted with two rounds of buoyancy purification.

### ix Transmission Electron Microscopy

For negatively stained TEM of purified GVs, samples were diluted to OD₅₀₀=0.2 in 1×PBS and loaded to 200-mesh carbon-coated copper grids (Ted Pella, Redding, CA) for 3 minutes. Excess liquid was carefully blotted away with filter paper. The samples were then stained with a 2% (w/v) solution of uranyl acetate (Electron Microscopy Sciences, Hatfield, PA). High-resolution TEM images were captured using a JEOL JEM-2010 TEM and JEOL JEM-2100 Field Emission Gun TEM.

### x Density Gradient Buffer Preparation

Density gradient buffer of 1.02 g/mL, 1.04 g/mL, 1.06 g/mL, and 1.08 g/mL were obtained by mixing flow buffer (1×PBS with 2% of BSA) with Cytiva Ficoll-Paque™ (Ficoll, Fisher Scientific) for a correct amount for each condition. These mixtures were stored at 4 C when not in use and tested against controls to ensure correct density values.

xi CD3+ T Cell Isolation from PBMCs via scFv-CD3-GV

For all cell studies, GVs were washed and concentrated to a final OD500=10. GVs were formulated in TBS and 2% BSA and stored at 4 °C for up to 1 month. Donor buffy coats were obtained through Gulf Coast Consortium.

Standard buffy coat isolation was done using the industry standard Ficoll procedure to obtain peripheral blood monocular cells (PBMCs). After isolation, PBMCs were counted, and the required number was added to the Eppendorf tube (2×106 cells in 50 μL of flow buffer). The cell suspension was incubated with 350 μL of scFv-CD3-GV solution (5 OD) for 1 h under 4 C and vigorous spinning.

Spin columns with collection tubes (Fisher Scientific) were utilized to establish the buoyancy-activated cell sorting system. The filters in the spin columns were removed in advance. 1 mL of density buffer was prepared as previously described and placed in each collection tube. The cell suspension and GV mixture was gently added to the spin column. The collection tube as well as the spin column were then centrifuged under 400 g for 30 min. Top fraction of the mixture was obtained by collecting the solution in the spin column, while the down fraction was the rest of the solution in the collection tube. Countess™ Cell Counting Chamber Slides (Invitrogen) were utilized to obtain cell number in top and down fractions.

xii CD3+ T Cell Isolation from PBMC via Streptavidin-GV

PBMCs were obtained and buoyancy-activated cell sorting system was established as previously described. After isolation, PBMCs were counted and the required number was added to the Eppendorf tube (2×106 cells in 50 μL of flow buffer). The cell suspension was incubated with 5 μL of Biotinylated Anti-Human CD3 (Thermo Fisher Scientific) for 45 min under 4 C. Then, the cell solution was spun at 400 g for 10 min to remove excess antibody. 400 μL of streptavidin-GV solution (5 OD) was added to the cell solution and incubated for 1 h under 4 C and vigorous spinning. The cell suspension and GV mixture was gently added to the spin column. The collection tube as well as the spin column were then centrifuged under 400 g for 30 min.

In some embodiments, buoyancy assisted cell sorting was accomplished with cells expressing a protein of interest (e.g. CD3, CD28). First, the cells expressing a protein of interest were incubated for 10 minutes at room temperature with the corresponding biotin-modified antibody. After incubation, streptavidin-modified gas vesicles were mixed into the sample and further incubated for 30 minutes to facilitate the biotin-streptavidin interaction at the cell surface. Labeled cell samples were then further processed as is or loaded on top of Percoll density media adjusted to a final solution density of about 1.01 g/mL, about 1.02 g/mL, about 1.03 g/mL, about 1.04 g/mL, about 1.05 g/mL, about 1.06 g/mL, about 1.07 g/mL, or about 1.08 g/mL to assist with separation of gas vesicle modified cell populations. The sample was then centrifuged for 10 minutes at 400×g, which generated a top fraction containing gas vesicle tagged cells and a bottom fraction containing unmodified cells. The top fraction was a floating cell layer that able to be manually isolated, or in one embodiment, may be captured in an insert that retains the liquid sample by surface tension where removal of the insert resulted in complete recovery of the top fraction.

xiii Cell Staining

The cell samples were collected and stained for flow cytometry. Cell samples were mixed with 10 μL of Fc Block (BD Bioscience) and incubated for 15 min under 4 C in advance. Later, 5 μL of desired antibodies were added to the mixture and incubated in the dark for 45 min under 4 C. Then, the cell solution was spun at 400 g for 10 min and the supernatant was discarded. Flow buffer was then added to the cell samples for flow cytometry analysis.

Cell samples were stained with CD3 (Alexa Fluor®700 Mouse Anti-Human CD3, BD Bioscience), CD4 (Alexa Fluor®647 Mouse Anti-Human CD4, BioLegend), CD8 (CD8 Monoclonal Antibody (3B5), PE-Texas Red, Invitrogen), CD14 (CD14 Monoclonal Antibody (61D3), PE-Cyanine5, Invitrogen), and 7-AAD (7-Aminoactinomycin D, Invitrogen).

### xiv Cell Type Analysis Via Flow Cytometry

All flow cytometry analysis was performed on the Sony MA3800 Cell Sorter. UltraComp eBeads™ (Invitrogen) were utilized to establish varying fluorescent libraries before the experiment. Desired antibodies were incubated the beads for 45 min under 4 C. Excess antibodies were removed by centrifuging the solution under 400 g for 10 min. The stained beads were then sent into the instrument and the fluorescent signal would be measured.

Prior to runs, the instrument was set up and standards were run to ensure correct working conditions. For each run, fluorescent controls and/or biological control were added to ensure proper gating. As for gating strategy, typically FSC vs SSC was gated to target the cell population in interest. Following that parent gate, FSH vs FSA was gated to exclude any doublets/triplets and show only single cell populations. Following that gate, a viability control (7-AAD) was used to gate for all viable, single cell populations. All flow cytometry analysis was done using FlowJo software and compared to same-run controls.

### B. Protein Purification for Industrial Enzyme Manufacturing

a. Creating High-Density Affinity Tags on GV Surface

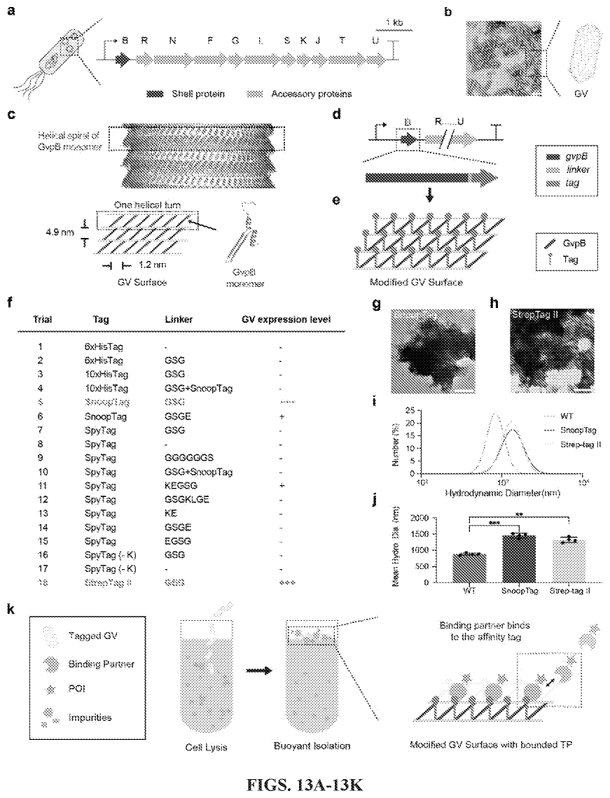

Previous research has demonstrated that the A. flos-aquae scaffolding protein GvpC from the genetically engineered GV construct, Acoustic reporter gene 1 (ARG1) (Bourdeau et al., 2018; Hurt et al., 2023), can be amenable to appending an affinity tag on either the N- or C-terminus (Lakshmanan et al., 2016). Additionally, GvpC from other different species can accommodate fusions with bacterial and viral polypeptides. However, GvpC was only sparsely present on the GV surface with an estimated ratio of 1:20 to the major shell proteins (Huber et al., 2023; Dutka et al., 2023). Consequently, by genetically appending affinity tags to GvpC, the availability of binding sites for the protein of interest (POI) on the surface of each GV particle becomes scant. Given this limited binding capacity, a substantial number of GVs would be required to bind and purify large amounts of the desired protein, undermining the objective of developing an economical and scalable approach. Therefore, GVs encoded by the pNL29 operon (FIG. 13A), originally cloned from Bacillus megaterium, were selected as they can be robustly expressed in E. coli and purified out (FIG. 13B) without the GvpC (Li & Cannon, 1998). Specifically, compositions comprising an affinity tag appended on the primary shell protein GvpB were designed and studied.

A recent study by Huber et al. demonstrated that the cylindrical shell of GVs in B. megaterium is constructed from GvpB monomers (Huber et al., 2023). These monomers are densely packed in a helical spiral, with a lateral spacing of 1.2 nm and a helical spacing of 4.9 nm, and ~93 monomers each helical turn (FIG. 13C) ((Huber et al., 2023). Since the shell protein is packed tightly in this highly crystalline fashion, altering the protein sequence of the major shell protein face considerable challenge. Mutating any parts of the shell protein increase both the risks of distorting the tight crystalline packing and the risks of interfering their interactions with the assembly factor proteins. However, they also demonstrated that the C-terminus of GvpB has a more limited role in stabilizing the assembly compared to the N-terminus (Huber et al., 2023). Additionally, the C-terminus is relatively flexible within the assembled GV shell and also exposed to the outer surface of GVs (Huber et al., 2023). Therefore, the C-terminus of GvpB, without being bound by theory, can be genetically modified without disrupting the assembly of GVs (FIG. 13D). If affinity tags are genetically appended to the C-terminus of GvpB, upon expression and assembly, the outer surface of the GVs could be densely covered with these affinity tags (FIG. 13E).

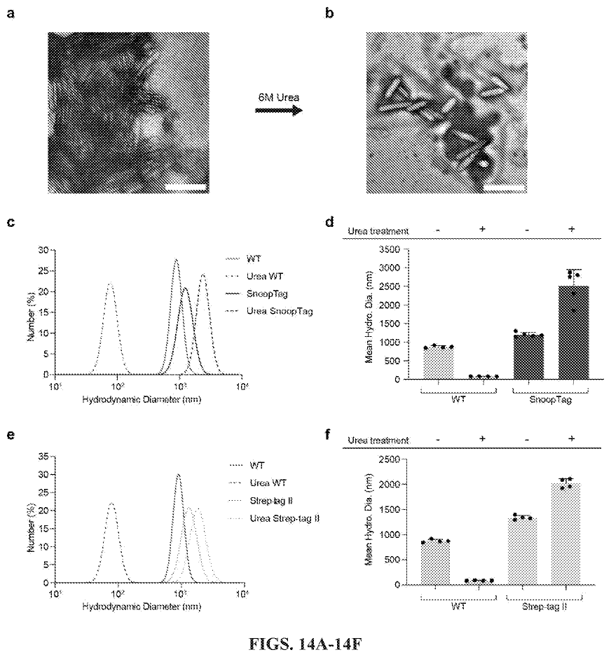

A range of fusions to the C-terminus of Mega GVs (FIG. 13F) that include various protein tags and linkers in between to further enhance the flexibility of C-terminus of GvpB was screened. After expression optimization, centrifugally assisted purification, the majority of the designs failed to produce a fully assembled GV, in agreement with expectations. However, two candidates emerged: SnoopTag (KLGDIEFIKVNK (SEQ ID NO:2)) and Strep-tag II (WSHPQFEK (SEQ ID NO:4)). After the purification process, these two tagged GVs variants underwent characterization through both transmission electron microscopy (TEM) and dynamic light scattering (DLS), with wildtype pNL29 GVs serving as the control group (FIGS. 13G-13J). Notably, like their wildtype counterparts, the tagged GVs also remained clustered post-purification from E. coli. However, a distinction that emerged from the DLS measurements (FIG. 13I, FIG. 13J) is that the clustering formed by the tagged GVs was larger in size than the wildtype GVs. The established protocol, which involves treatment with 6M urea (Lakshmanan et al., 2017), for unclustering wildtype pNL29 GVs (FIG. 14A, FIG. 14B), was found to be ineffective for the tagged GVs. Surprisingly, instead of reducing the hydrodynamic diameter of GVs by an order of magnitude, the size of the clusters increased after treatment with 6M urea (FIGS. 14C-14F).

Upon successful expression of the two tagged GVs in E. coli, the functional efficacy of high-density affinity tags on the GV surfaces was assessed. The objective was to efficiently bind these GVs to target proteins using the corresponding binding partners of the tags, followed by the buoyancy-driven isolation of the target proteins (FIG. 13K).

b. Buoyancy Isolation of Fluorescent Proteins

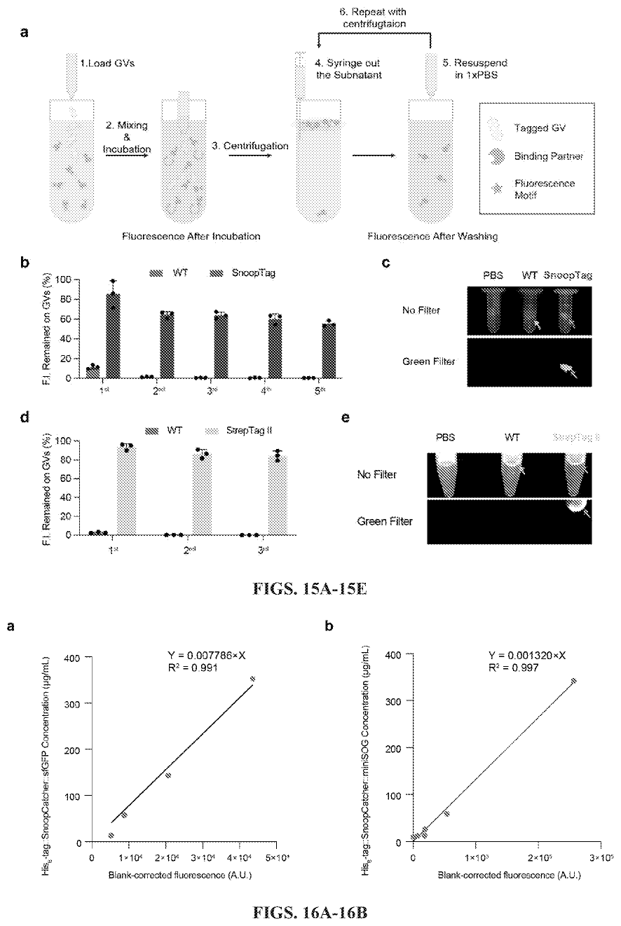

To establish the process design and obtain quantitative results of its performance, fluorescent proteins were chosen as POIs to increase ease of visualization and quantification of protein levels based on the fluorescence readouts (FIG. 15A, FIG. 16).

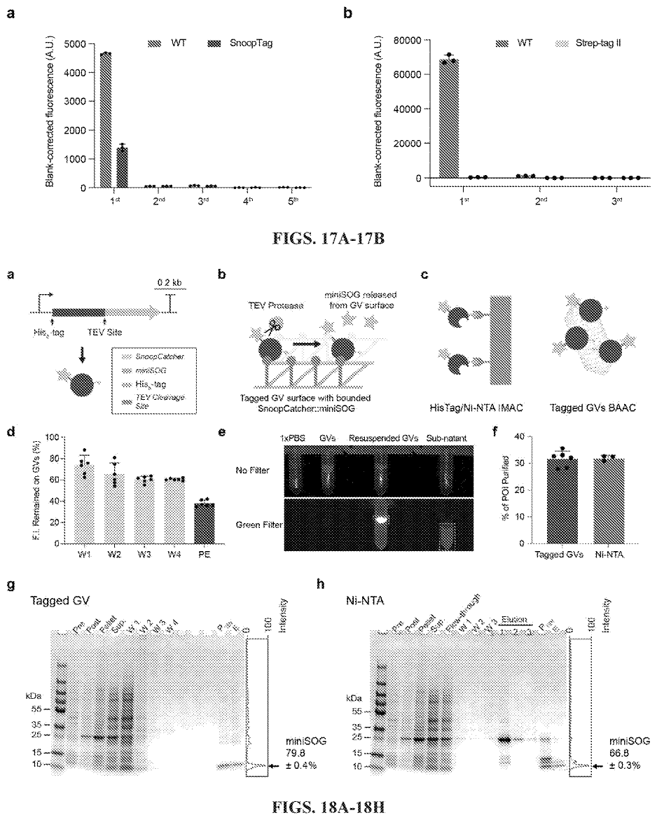

The process was started by mixing tagged GVs with fluorescence-labeled binding partners (purified His₆-tag::SnoopCatcher::superfolder Green Fluorescent Protein (sfGFP) for SnoopTag GVs and Alexa Fluor™ 488 streptavidin for Strep-tag II GVs) in tubes and incubated for an hour at room temperature. Post-incubation fluorescence intensity was measured (FIG. 15A). After centrifugation, floating GVs bound to labeled partners were isolated by removing the underlying liquid with a syringe (FIG. 15A). These GVs were resuspended in 1×PBS, and their fluorescence was measured again (FIG. 2a). This cycle continued until there was no significant change in fluorescence, and running at least three rounds of buoyancy isolation confirmed that the POIs were tightly bound to the GVs' surface, as demonstrated by the fluorescence readout (FIG. 15B, FIG. 15D). Also, direct fluorescence imaging the plastic tube revealed that POIs were isolated to the top of the tube as expected (FIG. 15C, FIG. 15E). In comparison, the WT GVs without the affinity tag pulled out minimal amount of nonspecific binding in the first round, which were nearly completely removed starting from the second round of buoyancy isolation (FIG. 15B, FIG. 15D). Direct fluorescence imaging also revealed that no POIs were bound to WT GVs following multiple rounds of buoyancy-driven isolation. Fluorescence readouts of the underlying liquid removed further corroborated that the majority of POIs were bound to tagged GVs rather than to WT GVs (FIG. 17).

The evidence provided above supports that GVs featuring surface-modified with SnoopTag or Strep-tag II can effectively interact with their respective binding partners. SnoopTag and SnoopCatcher were thus selected for further development.

c. Performance Comparison with Common Affinity Chromatography Methods

The purified tagged GVs were assessed as affinity substrate analogs to ‘resins’ or ‘beads’ commonly used in affinity chromatography for the purification of POIs. To this end, a fusion protein serving as the POI was designed by attaching miniSOG, a 12.3 kDa flavin-binding protein with dual capabilities for fluorescence and singlet oxygen photogeneration (Ruiz-Gonzalez et al., 2013; Qi el al., 2012), to the C-terminus of SnoopCatcher, which was already engineered with a His₆-tag at its N-terminus (FIG. 18A). Given the irreversible covalent bonding between SnoopTag and SnoopCatcher, a TEV protease cleavage site (ENLYFQ|S (SEQ ID NO:5)) was strategically incorporated between SnoopCatcher and miniSOG. This modification allows the detachment of miniSOG from GV surfaces upon incubation with TEV protease (FIG. 18A, FIG. 18B). Furthermore, the presence of the His₆-tag enables performance of a comparative analysis of tagged GVs buoyancy-assisted affinity chromatography (BAAC) with traditional His-tag/Ni-NTA immobilized metal affinity chromatography (IMAC) (FIG. 13C).

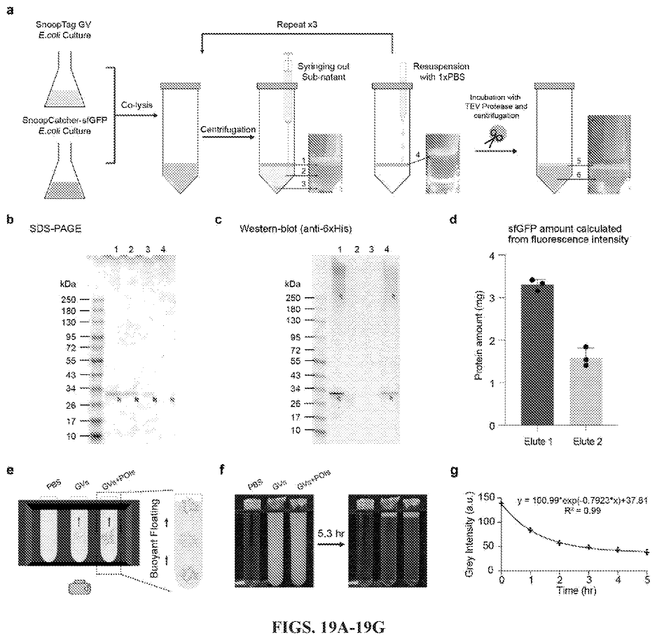

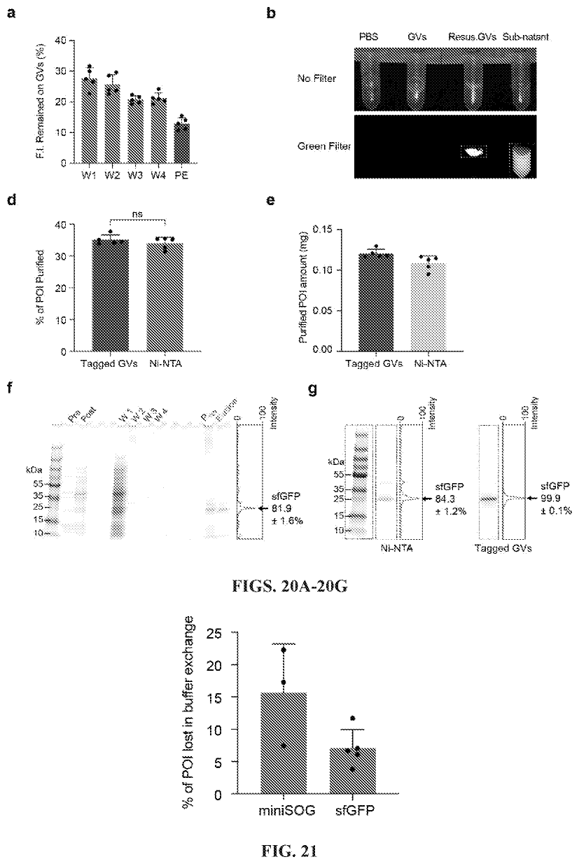

The amount of His₆-tag::SnoopCatcher::miniSOG was calculated based on the fluorescence readouts (FIG. 16B) to exactly meet the theoretical binding capacity of either Ni-NTA beads or tagged GVs. Adhering to the workflow outlined above (FIG. 18A), a the fluorescence readout of POIs on SnoopTag GVs was consistently stabilized after four rounds of buoyancy isolation (FIG. 19D), suggesting that the miniSOG version POIs are tightly bound to the surfaces of the GVs. Following incubation with TEV protease and subsequent buoyancy isolation to release miniSOG from the SnoopTag GVs, it was observed that approximately 60% of the initial fluorescence remained on the GVs (FIG. 19D). This result confirmed the successful partial release of miniSOG. Direct fluorescence imaging post-TEV protease treatment and buoyancy isolation revealed that a greater amount of miniSOG remained on the GV surface compared to what was released into the supernatant (FIG. 19E). One plausible explanation, without being bound by theory, for this observation could be that the tight clustering of GVs limits the accessibility of TEV protease to the GV surface. Despite partial releasing, the amount of miniSOG purified from clarified cell lysate using the BAAC was comparable to that obtained using Ni-NTA beads (FIG. 19F). Additionally, purification using tagged GVs yielded miniSOG with a higher purity level (79.8±0.4%) compared to Ni-NTA-based purification methods (66.8±1.9%), as shown by gel densitometry measurements of the elution fractions after the removal of TEV protease (FIG. 19G, FIG. 19H). Similarly, the consistency of these findings was also observed in the purification of sfGFP, further substantiating the general applicability of our method (FIG. 20). Additionally, a loss of POIs during the buffer exchange process used to remove imidazole in the His-tag/Ni-NTA IMAC (FIG. 21) was observed; however, this could have been prevented due to the consistent buffer used in the tagged GVs BAAC procedures.

d. Simple One-Step Purification Workflow

One key advantage of GV-based buoyancy isolation methods disclosed herein is removal of the need to prepare the affinity beads themselves. The users can simply grow the bacterial culture to produce the “beads”, i.e. engineered GVs, when they are raising their POIs. Then one can mix the two types of cells together, lyse them in a single pot, and continue through protein purification. Described below is the use of fluorescent proteins to test this simple, one-step purification workflow.

The one-step protein purification workflow commences with the simultaneous lysis of E. coli cultures of SnoopTag GVs and the POIs, His₆-tag::SnoopCatcher::sfGFP. The step is followed by a one-hour incubation at room temperature to enable the binding of tagged GVs to the POIs. After 30-minute centrifugation, SnoopTag GVs with bound POIs are separated from unbound POIs, impurities, and inclusion bodies through buoyancy isolation (FIG. 19A). The remaining steps, including multiple rounds of buoyancy purification and incubation with TEV protease, followed the protocols described above. Due to the similar molecular weights of sfGFP and commercial TEV protease, which features a His₆-tag, Western blotting was employed as an additional analytical tool to distinguish between them. When comparing the results of SDS-PAGE and Western blotting, the bands present in lanes 2 and 3 in the SDS-PAGE analysis were absent in the Western blot, confirming the complete removal of either TEV protease or the shell protein from tagged GVs, which were shown as smearing in lanes 1 and 4 of the Western blot results (FIG. 19B, FIG. 19C). Using this one-step buoyancy isolation approach, a yield of nearly 5 mg of protein per 50 mL E. coli culture was achieved (FIG. 19D).

e. Equipment-Free Purification

Next, an attempt was made to bypass centrifugation entirely, relying solely on normal gravity for protein isolation.

Wildtype GVs are nanoscale objects of a few hundred nanometer in diameter, and thus it is commonly needed to conduct at least several hours of centrifugation to isolate GVs (Lakshmanan et al., 2017). While the buoyancy force scales with the volume of the object and thus the diameter, increasing the overall particle size would present the most feasible method to increase the flotation speed. Fortunately, both types of engineered GVs were observed in clustered forms (FIGS. 13G-13J, FIG. 16).

To test GVs' flotation speed, a time-lapse photography of the tubes for a certain frame of time was set up (FIG. 19E). In both the tagged GVs without POIs and those bound with fluorescent proteins, approximately 5.3 hours were required for the majority of GV particles to ascend buoyantly to the 1×PBS surface (FIG. 19F). Since the solution's opacity can indicate the position of GVs, the intensity within a central region of interest (ROI) of the tube was measured hourly to quantify the flotation speed. The ROI grey intensity decreased over time, fitting a one-phase decay nonlinear model with a half-life of 0.87 hours (FIG. 19G). This suggests, without being bound by theory, that in a fixed-volume container, most clustered GV particles can ascend approximately 5 centimeters within an hour.

f. Discussion

The gas vesicles used in the presently disclosed methods are inaugural examples demonstrating that the primary shell protein of GVs can be genetically modified without compromising the effective expression and assembly of GVs in E. coli, underscoring the potential of genetically engineering GV surfaces with high density as a versatile platform for diverse applications. Furthermore, by demonstrating that GVs with affinity tags on their surface can function analogously to affinity resins for purifying POIs, the potential uses of GVs are expanded beyond biomedical engineering to biomanufacturing applications, especially the purification of protein biologics.

The successful appending of these two tag systems, Strep-tag II and SnoopTag, on GVs is significant in protein tagging research. Their complementary nature allows for a broad spectrum of applications in biotechnology and related disciplines. Specifically, the non-covalent nature of Strep-tag II offers the flexibility of conventional protein cargo release via small molecules through competitive binding, which can be invaluable when delicate modulation or targeted release of proteins is requisite (Voss, S. & Skerra, 1997; Schmidt et al., 2013). On the other hand, the covalent bond of SnoopTag (Veggiani et al., 2016) paves the way for innovative applications, opening doors to the potential of harnessing GV-bound proteins directly for enzyme catalysis, thereby pushing the boundaries of traditional enzyme applications. Furthermore, this covalency offers an opportunity to design controllable, self-cleaving protein or tag modules (Levitin et al., 2005; Li, 2011) that leverage stimuli-responsive mechanisms like pH or temperature-induced inteins (Wood et al., 1999; Zeidler et al., 2004) for meticulously regulated cleavage and activation.

In parallel, the presently disclosed methods offer a potential alternative to traditional affinity chromatography in the realm of protein purification. Specifically, the one-step buoyancy isolation using tagged GVs offers a “gentle” technique. Traditional methods often subject POIs to pelleting and change of buffer conditions, leading to potential inconsistencies such as incomplete removal of unwanted chemicals or loss of target proteins (Block et al., 2009; Burgess, 2018; Charcosset, 1998). In contrast, the methods disclosed herein ensure proteins remain in a consistent buffer condition, enhancing reproducibility and integrity of results highlighting its promise for broader biotechnological applications. Additionally, the results from equipment-free purification suggest that the presently disclosed methods hold promise for use in low-resource settings, particularly in the areas of biomanufacturing and diagnostics.

In summary, the present disclosure provides novel compositions and methods for industrial-scale purification of recombinant proteins that reduces reliance on affinity resins and columns. Furthermore, the high-density surface modification of gas vesicles offers potential use as an assembly platform (Oiseth et al., 2002; Farooq et al., 2020; Sato et al., 2016) for various nanotechnologies and biomaterial applications.

g. Methods

### i Cloning, Expression, and Purification of GVs

The plasmid pST39-pNL29 that encodes GV gene clusters from B. megaterium was obtained from Addgene (#91696). Oligos for molecular cloning were synthesized (Integrated DNA Technologies (IDT), Coralville, IA). All the GV variants with affinity tags appended on the C-terminus of gvpB were cloned into pST39 vector backbone using site-directed mutagenesis (SDM) (New England Biolabs (NEB), Ipswich, MA). For GV expression, plasmids were transformed in BL21 Star™ (DE3)pLysS One Shot™ E. coli strain (Thermo Fisher Scientific, Waltham, MA), which were then cultured in 50 mL LB Miller Broth (Thermo Fisher Scientific, Waltham, MA) with 25 μg/ml Chloramphenicol (MilliporeSigma, Burlington, MA), 100 μg/ml Carbenicillin (Gold Biotechnology, Olivette, MO), and 0.2% glucose (MilliporeSigma, Burlington, MA) in a 250 mL flask (Thermo Fisher Scientific, Waltham, MA). Cells were grown at 30 °C until the optical density at 600 nm (OD₆₀₀) reached 0.4, and then induced with 20 μM Isopropyl β-d-l-thiogalactopyranoside (IPTG) (Teknova, Hollister, CA) for 22 hrs at 30 °C. Cells were collected and pelleted by centrifugation at 400×g in 50 mL conical tubes for 6 hrs with a liquid depth ≤10 cm. The middle layer between the buoyant cells and the sedimented cells was removed and the remaining cells were mixed with 4 mL SoluLyse-Tris (Genlantis, San Diego, CA) per 50 ml culture and 250 μL/mL lysozyme (MilliporeSigma, Burlington, MA) and 10 μL/mL RNase-free Deoxyribonuclease I (DNase I, MilliporeSigma, Burlington, MA). After 30 min rotation at 4 °C, the lysate was transferred to 2 mL tubes and centrifuged for 2 hrs at 400 g at 4 °C. The buoyant gas vesicles (GVs) located at the top of the liquid surface were isolated by removing the liquid and cell pellets underneath them using a syringe. The isolated GVs were then washed by resuspending them in 1×PBS (Teknova, Hollister, CA) followed by centrifugally-assisted flotation for three cycles.

### ii Transmission Electron Microscopy

For negatively stained TEM of purified GVs, samples were diluted to OD₅₀₀=0.2 in 1×PBS and loaded to 200-mesh carbon-coated copper grids (Ted Pella, Redding, CA) for 3 minutes. Excess liquid was carefully blotted away with filter paper. The samples were then stained with a 2% (w/v) solution of uranyl acetate (Electron Microscopy Sciences, Hatfield, PA). High-resolution TEM images were captured using a JEOL JEM-2010 TEM and JEOL JEM-2100 Field Emission Gun TEM.

### iii Hydrodynamic Size Measurement

The hydrodynamic diameters of all GV variants were measured using a Malvern Zen 3600 Zetasizer (Malvern, UK). Purified GV samples were diluted to an OD₅₀₀=0.2 in 1×PBS. Then, 500 μL of each sample were transferred into a cuvette (Thermo Fisher Scientific, Waltham, MA). A minimum of three readings per sample were acquired, and each GV variant had at least three biological duplicates.

### iv Cloning, Expression, and Purification of Targeting Protein

The open reading frame of SnoopCatcher::sfGFP and SnoopCatcher::miniSOG were cloned into the pST39 vector with a N-terminal His₆-tag. The transformed cells were grown at 30 °C until OD₆₀₀ reached 0.6 and then induced with 400 mM IPTG for 20 hrs at 20 °C. Cells were harvested, resuspended in lysis buffer with 50 mM Tris-HCl, 300 mM NaCl, 10 mM imidazole and pH of 7.5. Cells were lysed by 5 cycles of freeze-thaw followed by 5 cycles of probe sonication. DNase I, Benzonase® Nuclease (MilliporeSigma, Burlington, MA) and Dithiothreitol (DTT) were added at 20 μg/mL, 5000 units/mL, and 1 mM, respectively, followed by 30 min incubation at 37 °C. Next, the samples were centrifuged for 30 min at 24,000×g and the supernatant was applied for downstream experiments.

Next, to acquire purified fluorescent proteins, the supernatant was loaded into a Ni-NTA affinity chromatography (Qiagen USA, Germantown, MD). After washing at 20 mM imidazole, the target protein was eluted with 300 mM imidazole. Fractions containing the target protein were buffer exchanged with 1×PBS and stored at −80 °C

### v SDS-PAGE and Protein Purity Quantification

SDS-PAGE was performed using 4-15% Mini-PROTEAN™ TGX Stain-Free™ Protein Gels (Bio-Rad Laboratories, Hercules, CA). Samples were mixed with a final concentration of 1×Laemmli Sample Buffer (Bio-Rad Laboratories, Hercules, CA), and 10 mM Dithiothreitol (DTT) was added to reduce samples. SDS-PAGE gels were run at 120 V in 25 mM Tris, 192 mM glycine, 0.1% (w/v) SDS, pH 8.3. Gels were stained with Coomassie Brilliant Blue G-250 stain (Bio-Rad Laboratories, Hercules, CA), destained with homemade destaining buffer (1:1:8 vol % of methanol, glacial acetic acid H₂O), and imaged using the FluorChem M imager. Gels' band intensity profiles were analyzed with Fiji (ImageJ2) by plotting the profile of the selected lane (Wash in FIG. 19). Percentage purity was defined as 100×(target protein band intensity in the lane/all protein bands intensity in the lane)

### vi Protein Fluorescence Intensity to Concentration Conversion Standard

Purified SnoopCatcher fused fluorescent proteins were serial diluted with 1×PBS. The fluorescence intensity (His₆-tag::SnoopCatcher::sfGFP: excitation=485, emission=510 for; His₆-tag::SnoopCatcher::miniSOG: excitation=447, emission=501) of 150 μL dilutions in 96 well plates were measured (Corning® 96 Well CellBIND® Microplates, Corning) using a microplate reader (Agilent BioTek Synergy H4 Hybrid, Agilent).

The concentrations of those same dilutions were measured followed the protocol provided by Pierce™ Modified Lowry Protein Assay Kit (Thermo Fisher Scientific, Waltham, MA).

### vii Functionality Test of Affinity Tags on GVs

Both purified WT Mega GVs and SnoopTag GVs were diluted to the optical density at 500 nm (OD₅₀₀) equal to 0.5 by 1×PBS with a total volume of 800 μL in 2.0 mL microcentrifuge tubes. These GVs were loaded into a 200 μL solution of purified His₆-tag::SnoopCatcher::sfGFP at a concentration of 345 μg/mL, followed by pipette mixing. These mixture samples were then subjected to rotary incubation at room temperature for 1 hour. After incubation, the fluorescence intensity readings of samples were acquired in the same way mentioned above. Subsequently, samples were centrifuged for 10 min at 400×g to separate the GVs with bounded target protein and sub-natant liquid with unbounded ones. The buoyant GVs-target protein complex at the top of the liquid surface were isolated by removing the liquid underneath them using a syringe. The complex samples were then washed by resuspending in 1000 μL 1×PBS, followed by measuring the fluorescence intensity. The centrifugation-resuspension cycles were repeated 5 times in total as shown in FIG. 18A.

Purified Strep-tag II GVs and WT Mega GVs were diluted to OD₅₀₀=1.0 by 1×PBS with a total volume of 1000 μL in 1.5 mL microcentrifuge tubes. 10 μL of 2 mg/mL Alexa Fluor™ 488 streptavidin (Thermo Fisher Scientific, Waltham, MA) was introduced into each GV sample. The sample incubation, fluorescence intensity measurement, and centrifugation-resuspension cycles were processed in the similar way as for SnoopTag GVs. The fluorescence intensity measurement was adjusted at excitation=495, emission=519 for Alexa Fluor™ 488. The centrifugation-resuspension cycles were repeated three times. The percentage of fluorescence intensity remained on GV after each wash was calculated by 100×(Fluorescence intensity after each resuspension washing/Fluorescence intensity after incubation). A FluorChem M imager was used to visualize the fluorescence among samples by setting two channels with the same exposure time (2.4 s): channel 1 with epi white light and no filter (no filter) and channel 2 with blue light and green filter (green filter).

viii Side-by-Side Protein Purification Comparison

50 μL of Ni-NTA magnetic bead slurry (Biolabs, 2020) (New England Biolabs (NEB), Ipswich, MA) with a binding capacity of 7.5 mg/mL was used as a counterpart for side-by-side protein purification comparison with SnoopTag GVs with the OD₅₀₀ of 12. The fluorescence intensity of the His₆-tag::SnoopCatcher::miniSOG lysis supernatant was measured using the aforementioned methods to estimate target protein concentration. The lysis supernatant, containing a target protein amount sufficient to saturate the binding capacities of both SnoopTag GVs (500 μL) and Ni-NTA magnetic bead (700 μL), was loaded into each sample accordingly. For the Ni-NTA magnetic bead, the provided protocol was followed, included 1 hr, 4 °C incubation, collection of flow-through, three washing steps, and three elution steps with all steps fluorescence intensity measured. Samples from elution steps were undergone buffer exchange against 1×PBS by using MilliporeSigma™ Amicon™ Ultra-0.5 Centrifugal Filter Units (MilliporeSigma, Burlington, MA) with a 10 kDa molecular weight cutoff membrane. Purified His₆-tag::SnoopCatcher::miniSOG were incubated with 30 μL TEV Protease with His₆-tag (New England Biolabs (NEB), Ipswich, MA) at 30 °C for 12 hr, followed by extra Ni-NTA magnetic bead slurry to remove His₆-tag::SnoopCatcher and TEV Protease.

For SnoopTag GVs, fluorescence intensity and OD₅₀₀ of the samples were measured after a 1 hr incubation with the lysis supernatant at 4 °C. Subsequently, samples were centrifuged and washed with 1×PBS three times, following the same procedures as mentioned in the previous section. The TEV Protease procedures for GVs with bounded His₆-tag::SnoopCatcher::miniSOG were the same as in Ni-NTA magnetic bead purification. The measurements of fluorescence intensity and OD₅₀₀ were acquired after each procedure. Samples collected from each step for both purification methods were performed SDS-PAGE and analyzed as mentioned in early section.

ix Co-Lysis of SnoopTag GVs and SnoopCatcher::sfGFP E. coli Culture

Both cultures, each with a volume of 50 mL, were grown and collected following the same procedures as previously mentioned. GVs culture was centrifuged at 400×g for 6 hr, followed by removing the middle layer between the buoyant cells and the sedimented cells to a final volume of 10 mL. SnoopCatcher::sfGFP culture was centrifuged at 3000×g for 15 min, followed by discarding the super-natant and transferring the sedimented cells into 10 mL GVs culture. Cells from two cultures were co-lysed by 4 mL SoluLyse-Tris (Genlantis, San Diego, CA), 90 U/mL Benzonase® endonuclease with 1 mM Mg²⁺ (MilliporeSigma, Burlington, MA), 0.5 mg/mL lysozyme (MilliporeSigma, Burlington, MA), and 100 μL Halt™ Protease Inhibitor Cocktail (Thermo Fisher Scientific, Waltham, MA). After 30 min incubation at 37 °C, the whole lysed culture was centrifuged at 400×g for 10 min to isolate GVs with bounded target proteins, followed by removing underneath liquid and inclusion body. GVs were resuspended by 1×PBS and undergone the same three buoyancy isolation washes as before. The TEV Protease procedures were also perform as indicated in the last section, as well as the fluorescence intensity and OD₅₀₀ of each step. Samples after TEV protease procedures were collected and performed SDS-PAGE and Western blotting. His₆-tag Monoclonal Antibody (HIS.H8), HRP (Thermo Fisher Scientific, Waltham, MA) was used to label proteins in Western blotting; and bands were visualized by applying Pierce™ ECL Western Blotting Substrate (Thermo Fisher Scientific, Waltham, MA).

### x Time-Lapse Photography for GVs' Buoyant Floatation Speed Analysis

Two SnoopTag GVs samples were prepared with an initial OD₅₀₀=46 in 1×PBS with a total volume of 1 mL. One GVs sample was loaded with 96 μL of 32 mg/mL His₆-tag::SnoopCatcher::sfGFP, meanwhile another one was loaded with 96 μL 1×PBS. Both samples were incubated under rotation at room temperature for 1 hr, then transferred into transparent Falcon® 14 mL Round Bottom Polystyrene Test Tubes (Corning Inc., Corning, NY) and topped up with 1×PBS to reach a final volume of 12 mL. Alongside another tube containing 12 mL of 1×PBS, the samples were placed in a homemade 3D printed rack. Photos of the samples were taken every 20 seconds for unclustered GVs and every 30 seconds for clustered GVs, continuing until the end of the photography session. Photos of the samples were converted to 8-bit and the grey intensity of selected regions of interest (ROI) was analyzed using ImageJ.

### xi Quantification and Statistical Analysis

The statistical information of the experiments, including sample size (n), and P-values, can be located in the figures, captions for the figures, and the method section. The statistical analysis was carried out using GraphPad Prism software and is represented as the mean±standard deviation (STD). For all the multiple comparisons, the Welch and Brown-Forsythe ANOVA tests were performed, which is a version of one-way ANOVA that does not assume that all the groups were from a population with equal variances. p<0.05 was set as significantly different.

### REFERENCES

The following references, to the extent that they provide exemplary procedural or other details supplementary to those set forth herein, are specifically incorporated herein by reference:
Bashor et al., Nature Reviews Drug Discovery 2022, 21 (9), 655-675.
Biolabs, N. E. NEBExpress Ni-NTA Magnetic Beads (NEB #S1423). (2020).
Block et al., Methods in enzymology 463, 439-473 (2009).
Bourdeau et al., Nature 553, 86-90 (2018).
Burgess, Methods in enzymology 463, 331-342 (2009).
Burgess, Protein expression and purification 150, 81-85 (2018).
Campbell et al., Cancer research, 51(19), 5329-5338, (1991).
Cheung & Gu, International Biodeterioration & Biodegradation 59, 8-15 (2007).
Chakroun et al., Process Biochemistry 45, 507-513 (2010).
Charcosset, Journal of Chemical Technology & Biotechnology: International Research in Process, Environmental AND Clean Technology 71, 95-110 (1998).
Choe et al., Materials, 9(12):994, (2016).
DeMonte et al., Proteins, 81(9):1621-33, 2013.
Deutscher, Guide to protein purification. Vol. 182 (Gulf Professional Publishing, 1990).
Dos Santos et al., Biotechnology Advances 35, 41-50 (2017).
Dutka et al., Structure 31, 518-528 e516 (2023).
Farooq et al., Biosensors and Bioelectronics 157, 112163 (2020).
Flickinger, Downstream industrial biotechnology: recovery and purification. (John Wiley & Sons, 2013).
Franzreb et al., Applied microbiology and biotechnology 70, 505-516 (2006).
Frenea-Robin & Marchalot, Magnetochemistry 2022, 8 (1), 11.
Ghosh, Journal of Chromatography A 952, 13-27 (2002).
Gomari et al., Biotechnology advances 45, 107653 (2020).
Hanke & Ottens, Trends in biotechnology 32, 210-220 (2014).
Harrison, Protein purification process engineering. Vol. 18 (CRC Press, 1993).
Hu et al., Frontiers in Cell and Developmental Biology 2016, 4.
Huber et al., Cell 186, 975-986 e913 (2023).
Hurt et al., Nat Biotechnol 41, 919-+(2023).
Janson, Protein purification: principles, high resolution methods, and applications. (John Wiley & Sons, 2012).
Jozala et al., Brazilian journal of microbiology 47, 51-63 (2016).
Keeble et al., PNAS, 116(52):26523-33, 2019.
Korndofer, Protein Sci., 11(4):883-893, 2002.
Labrou, Protein Downstream Processing: Design, Development and Application of High and Low-Resolution Methods, 3-10 (2014).
Lakshmanan et al., Nat Protoc 12, 2050-2080 (2017).
Lakshmanan et al., Acs Nano 10, 7314-7322 (2016).
Leptihn et al., Nature protocols 8, 1048-1057 (2013).
Levitin et al., Journal of Biological Chemistry 280, 33374-33386 (2005).
Li, Biotechnology letters 33, 869-881 (2011).
Li & Cannon, Journal of bacteriology 180, 2450-2458 (1998).
Linhult et al., Protein and peptide letters 12, 305-310 (2005).
Lipsitz et al., Nature Biotechnology 2016, 34 (4), 393-400.
Liou et al., PLOS ONE 2015, 10 (5), e0125036.
Liu et al., Advances in Colloid and Interface Science 284, 102254 (2020).
Miltenyi et al., Cytometry, 11 (2), 231-238, (1990).
Nechushtan et al., J. Biol. Chem., 272(17):11597-11603 (1997).
Øiseth et al., Applied surface science 202, 92-103 (2002).
Onda et al., Cancer Research, 64(4):1419-1424 (2004).
Palani et al., Bone Marrow Transplantation, 58(2):160-167, (2023).
Peydayesh & Mezzenga, Nat Commun 12, 3248 (2021).
Pfeifer, Nat Rev Microbiol 10, 705-715 (2012).
Qi et al., Proceedings of the National Academy of Sciences 109, 7499-7504 (2012).
Rathore et al., Biotechnology letters 40, 895-905 (2018).
Roe, Protein purification applications: a practical approach. Vol. 245 (OUP Oxford, 2001).
Ruiz-González et al., Journal of the American Chemical Society 135, 9564-9567 (2013).
Sato et al., Composites Part A: Applied Science and Manufacturing 83, 72-79 (2016).
Schmidt et al., Protein expression and purification 92, 54-61 (2013).
Scopes, Protein purification: principles and practice. (Springer Science & Business Media, 1993).
Turner et al., Purinergic Signalling, 16(3):389-401, (2020).
Veggiani et al., Proceedings of the National Academy of Sciences 113, 1202-1207 (2016).
Viswanath et al., Enzyme research 2014 (2014).
Voss & Skerra, Protein engineering 10, 975-982 (1997).
Wang et al., Nature Biomedical Engineering 2022.
Wang et al., Nature Biomedical Engineering 2023.
Walsby, Microbiol Rev 58, 94-144 (1994).
Weil et al., Regenerative Medicine 2017, 12 (4), 397-417.
Weitman et al., Cancer research, 52(12), 3396-3401 (1992).
Winkles, J. A., Nature reviews Drug discovery, 7(5), 411-425, (2008).
Winthrop et al., Clinical Cancer Research, 9(10 Pt 2):3845S-3853S, (2003).
Wood et al., Nat Biotechnol 17, 889-892 (1999).
Yang & Sagis, Current Opinion in Colloid & Interface Science 56, 101499 (2021).
Zeidler et al., Nat Biotechnol 22, 871-876 (2004).

What is claimed:

1. A method of purifying a target protein from a sample comprising:
(A) obtaining a collection of labeled gas vesicle compositions, wherein gas vesicles comprising one or more proteins on the surface of the gas vesicles and wherein the one or more proteins have been modified with one or more affinity tags to form the labeled gas vesicle composition; and
(B) contacting the collection of labeled gas vesicle compositions with a sample containing the target protein for a first time period; wherein the first time period is sufficient for the labeled gas vesicle composition and the target protein to bind to form a captured target protein-gas vesicle composition; and
(C) allowing the components of the sample containing the captured target protein-gas vesicle composition to equilibrate for a second time period; and
(D) isolating the captured target protein-gas vesicle composition from the sample mixture.

2. The method of claim 1, wherein the captured target protein-gas vesicle composition forms a floating layer in the sample mixture.

3. The method of claim 2, wherein the floating layer forms during the first or second time period.

4. The method according to any one of claims 1-3, wherein the target protein is on the surface of a cell type in the sample mixture.

5. The method of claim 4, wherein the cell is a lymphatic cell.

6. The method of either claim 4 or claim 5, wherein the cell is a T cell.

7. The method according to any one of claims 4-6, wherein the cell density of the captured target protein-gas vesicle composition is lower than the cell density of the unbound cell.

8. The method according to any one of claims 4-7, wherein the cell density of the captured target protein-gas vesicle composition is lower than the cell density of the unbound cell by between about 0.01 g/cm³ and about 0.04 g/cm³.

9. The method according to any one of claims 4-8, wherein the cell density of the captured target protein-gas vesicle composition is lower than the cell density of the unbound cell by about 0.04 g/cm³.

10. The method according to any one of claims 4-9, wherein the cell is derived from a patient.

11. The method according to any one of claims 1-10, wherein the target protein is present on a single cell type in the sample mixture.

12. The method according to one of claims 1-10, wherein the target protein is present on two or more cell types in the sample mixture.

13. The method according to any one of claims 1-12, wherein the collection of labeled gas vesicle compositions comprises two distinct sets of labeled gas vesicle compositions; wherein
(i) the first set of labeled gas vesicle compositions comprise a first affinity tag;
(ii) the second set of labeled gas vesicles compositions comprise a second affinity tag; and
wherein the first affinity tag and the second affinity tag are complementary.

14. The method of claim 13, wherein the labeled gas vesicle compositions further comprise a cell-specific marker.

15. The method of claim 14, wherein the cell-specific marker is attached to an affinity tag identical to the first affinity tag of the first set of labeled gas vesicle compositions.

16. The method according to any one of claims 1-15, wherein the contacting the labeled gas vesicle compositions with the sample mixture occurs in two steps, wherein
(i) the first step comprises contacting the first set of labeled gas vesicle compositions with the sample mixture; and
(ii) the second step comprises contacting the second set of labeled gas vesicle compositions with the sample mixture.

17. The method of claim 16, wherein the second set of labeled gas vesicle compositions is present in excess relative to the first set of labeled gas vesicle compositions.

18. The method according to any one of claims 1-17, wherein at least one of the affinity tags is a peptide or protein.

19. The method of claim 18, wherein the at least one of the affinity tags is a peptide.

20. The method of claim 19, wherein the at least one of the affinity tags is a protein.

21. The method according to any one of claims 1-20, wherein at least one of the affinity tags is an antibody.

22. The method of claim 21, wherein the antibody is an antibody against the target protein.

23. The method of claim 22, wherein the target protein is expressed on the surface of a cell.

24. The method according to any one of claims 21-23, wherein the antibody is an anti-CD3 antibody.

25. The method according to any one of claims 21-23, wherein the antibody is an anti-CD28 antibody.

26. The method according to any one of claims 1-25, wherein at least one of the affinity tags is streptavidin.

27. The method according to any one of claims 1-26, wherein at least one of the affinity tags is biotin.

28. The method according to any one of claims 1-27, wherein at least one of the affinity tags is SnoopTag.

29. The method according to any one of claims 1-28, wherein at least one of the affinity tags is SnoopCatcher.

30. The method according to any one of claims 1-29, wherein at least one of the affinity tags is SpyTag.

31. The method according to any one of claims 1-30, wherein the labeled gas vesicle composition and the target protein bind to form a covalent bond.

32. The method according to any one of claims 1-30, wherein the labeled gas vesicle composition and the target protein bind to form a non-covalent bond.

33. The method according to any one of claims 1-32, wherein the first time period is from about 15 minutes to about 5 hours.

34. The method according to claim 33, wherein the first time period is about 1 hour.

35. The method according to any one of claims 1-34, wherein the second time period is from about 15 minutes to about 5 hours.

36. The method of claim 35, wherein the second time period is about 1 hour or about 2 hours.

37. The method of claim 35, wherein the second time period is about 5 hours.

38. The method according to any one of claims 1-37, wherein the method does not comprise centrifugation.

39. The method according to any one of claims 1-37, wherein isolating the captured target protein-gas vesicle composition further comprises centrifugation.

40. The method according to claim 39, wherein centrifugation forms a concentrated layer of captured target-protein gas vesicle composition.

41. The method according to either claim 39 or claim 40, wherein the centrifugation is traditional centrifugation.

42. The method according to either claim 39 or claim 40, wherein the centrifugation is density-gradient centrifugation.

43. The method according to any one of claims 39-42, wherein the centrifugation is at 400×g.

44. The method according to any one of claims 1-43, wherein isolating the captured target protein-gas vesicle composition further comprises elutriation.

45. The method according to any one of claims 1-44, wherein the method further comprises separating the target protein from the captured target protein-gas vesicle composition following isolation from the sample mixture.

46. The method of claim 45, wherein the separation occurs via cleavage by a protease.

47. The method of claim 46, wherein the protease is TEV protease.

48. The method of claim 45, wherein the separation occurs via intein self-splicing.

49. The method of claim 48, wherein the intein is AI-CM mini intein.

50. The method of claim 49, wherein the intein self-splices upon pH change.

51. The method of claim 50, wherein the intein self-splices upon changing the pH from about pH 4 to about pH 7.

52. The method of claim 49, wherein the intein self-splices upon temperature change.

53. The method according to any one of claims 1-52, wherein the method further comprises detecting the presence of the target protein.

54. The method of claim 53, wherein the detection is a detection of the presence of a cell.

55. The method according to any one of claims 1-54, wherein the sample containing the target protein has a volume of about 10 L or greater.

56. The method according to any one of claims 1-55, wherein the sample containing the target protein has a volume of about 100 L or greater.

57. The method according to any one of claims 1-56, wherein the sample containing the target protein has a volume of about 1000 L or greater.

58. The method according to any one of claims 1-57, wherein the sample containing the target protein has a volume of about 10,000 L.


SEQUENCE LISTING (6 sequences)

SEQ ID NO: 1 - protein, 15 aa, synthetic construct
GSGKLGDIEFIKVNK

SEQ ID NO: 2 - protein, 12 aa, synthetic construct
KLGDIEFIKVNK

SEQ ID NO: 3 - protein, 11 aa, synthetic construct
GSGWSHPQFEK

SEQ ID NO: 4 - protein, 8 aa, synthetic construct
WSHPQFEK

SEQ ID NO: 5 - protein, 7 aa, synthetic construct
ENLYFQS

SEQ ID NO: 6 - protein, 10 aa, synthetic construct
RNFGLSKJTU